(19) United States
(12) Patent Application Publication
(10) Pub. No.: US 2012/0091884 A1
(43) Pub. Date: Apr. 19, 2012

(54) HEPTAAZAPHENALENE DERIVATIVES AND USE THEREOF IN ORGANIC ELECTROLUMINESCENT DEVICE

(75) Inventors: James Matthew Macdonald, Victoria (AU); Raju Adhikari, Victoria (AU); Almar Postma, Victoria (AU); Tadahiko Hirai, Victoria (AU); Kazunori Ueno, Victoria (AU)

(73) Assignee: Commonwealth Scientific and Industrial Research Organisation, Campbell, Australian Capital Territory (AT)

(21) Appl. No.: 13/257,431

(22) PCT Filed: May 21, 2010
(86) PCT No.: PCT/AU2010/000614

§ 371 Date: Dec. 1, 2011

(30) Foreign Application Priority Data
May 22, 2009 (AU) 2009902317
Nov. 27, 2009 (AU) 2009905825

(51) Int. Cl. H05B 33/14; C07D 487/16

(52) U.S. Cl. 313/504; 544/198; 544/209

(57) ABSTRACT

The present invention relates to an organic electroluminescent device comprising a pair of electrodes forming an anode and cathode, and one or more layers of organic compound arranged between the pair of electrodes, wherein the organic compound layer comprises heptaazaphenalene derivatives of formula (1). The present invention also relates to the said compounds.

### FIELD OF THE INVENTION AND RELATED ART

The present invention relates to organic electroluminescent devices, which are sometimes otherwise referred to as organic light emitting diodes (OLED). Organic electroluminescent devices emit light on the application of an electric field to one layer or multiple layers of an organic compound (organic material layer). The present invention also relates to organic compounds suitable for use in such devices.

An organic electroluminescent device is generally comprised of a pair of electrodes forming an anode and a cathode, and one layer or multiple layers comprising a hole injection layer, emission layer (with either fluorescent or phosphorescent material) and electron transporting layer. Into the organic layer(s), holes and electrons are injected from the anode and the cathode, respectively, thus resulting in excitons within the emission material. When the excitons transition to ground state, the organic luminescence device emits light.

According to the first study by Eastman Kodak Co. (“Appl. Phys. Lett”, vol. 51, pp. 913 (1987), an organic electroluminescent device which comprised a layer of an aluminium quinolinol complex (as electron transporting and luminescent material) and a layer of a triphenylamine derivative (as a hole transporting material) resulted in luminescence of about 1,000 cd/m² under an application of a voltage of 10 V. Examples of related U.S. Patents include U.S. Pat. Nos. 4,539,507; 4,720,432 and 4,885,211.

Further studies by Baldo et al. revealed a promising OLED using phosphorescent material as dopant. The quantum yield of the phosphorescent OLED was significantly improved (U.S. Pat. No. 6,830,828).

In addition to the above-mentioned OLED, polymer organic electroluminescent device (PLED) using a conjugated polymer material has been reported by a group from Cambridge University (Nature, vol. 347, pp. 539-(1990), U.S. Pat. Nos. 5,247,190; 5,514,878 and 5,672,678).

PLED has an advantage in terms of device fabrication as a printing methodology may be adopted for soluble polymer materials.

Although in the past twenty years OLED and PLED have shown significant progress in their performance, there still remain problems that need to be solved.

For instance, organic electroluminescent devices described above still show insufficient performance in terms of durability when used for a long time. The performance of organic electroluminescent devices can be further improved by studying new materials such as hole injection materials, hole transporting materials, host materials, emission materials and some others. Additionally, improvement of the device fabrication process is required.

An important consideration to improving organic electroluminescent device performance is to further decrease the driving current within the device in order to enhance device lifetimes. For example, some materials that address this concern have been proposed (U.S. Pat. No. 6,436,559, JP 3571977, JP 3614405). Despite these advances in organic electroluminescent devices using such materials, further improvements regarding stability and the performance are still required.

### SUMMARY OF THE INVENTION

A present invention provides improvements to the problems encountered in organic electroluminescent devices as mentioned above, or provides a useful alternative.

Specific embodiments may provide an organic electroluminescent device with high efficiency and longer life time.

Specific embodiments may also provide a stable device which has reduced current leakage at low current range.

According to the present invention, there is provided organic electroluminescent device comprising:
a pair of electrodes comprising an anode and a cathode, and
one or more layers of organic compound arranged between the pair of electrodes, wherein the organic compound layer, or one or more of the organic compound layers, comprises a compound represented by the following formula (1):

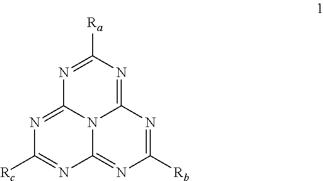

wherein:

Rₐ is selected from NR₁R₂, CR₇R₈R₉, Ar₁₆ and cyano;

R_{b} is selected from NR₃R₄, CR₁₀R₁₁R₁₂, Ar₁₇ and cyano;

R_{c} is selected from NR₅R₆, CR₁₃R₁₄R₁₅, Ar₁₈ and cyano;

R₁ to R₆, which may be the same or different, are each independently selected from the group consisting of substituted or unsubstituted aryl group, substituted or unsubstituted heterocyclic group, substituted or unsubstituted alkyl group and cyano, or each pair of substituents R₁ and R₂, R₃ and R₄ and/or R₅ and R₆ may together form a substituted or unsubstituted heterocyclic group incorporating the attached nitrogen atom, and wherein one or more of R₁ to R₆ may comprise a substituent containing a monomer or polymer chain;

R₇ to R₁₅ which may be the same or different, are each independently selected from the group consisting of hydrogen atom, halogen atom, nitro group, ketone group, amide group, cyano group, carboxylate group, sulfonate group, substituted or unsubstituted aryl group, substituted or unsubstituted alkyl group and substituted or unsubstituted heterocyclic group, or pairs of substituents R₇ and R₈, R₁₀ and R₁₁ and/or R₁₃ and R₁₄ may together form a substituted or unsubstituted cyclic group, in which case each of R₉, R₁₂ and R₁₅ is as defined previously or is absent; and in which any substituent on any of groups R₇ to R₁₅ may contain a monomer or polymer chain; and

Ar₁₆ to Ar₁₈ are each selected from substituted and unsubstituted aryl groups, in which the substituent may contain a monomer or polymer chain.

By using the organic compound as represented by formula (1) shown above in the organic electroluminescent device, the device can be driven with a low voltage and can have a long durability with high luminescence.

In some embodiments, the organic compound as represented by formula (1) can be used as either a hole injection layer or as a dopant in a hole transporting layer, or may be doped with a dopant to function as a hole transporting layer.

Furthermore, the layer or layers comprising the organic compound shown by formula (1) can be prepared by a variety of techniques including but not limited to vacuum deposition process, wet coating process and casting process.

The present invention also provides novel compounds within the range represented by formula (1):

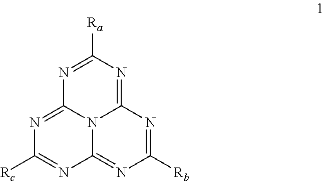

wherein:

Rₐ is selected from NR₁R₂, CR₇R₈R₉, Ar₁₆ and cyano;

R_{b} is selected from NR₃R₄, CR₁₀R₁₁R₁₂, Ar₁₇ and cyano;

R_{c} is selected from NR₅R₆, CR₁₃R₁₄R₁₅, Ar₁₈ and cyano;

R₁ to R₆, which may be the same or different, are each independently selected from the group consisting of: substituted or unsubstituted aryl group, substituted or unsubstituted heterocyclic group, substituted or unsubstituted alkyl group and cyano, or each pair of substituents R₁ and R₂, R₃ and R₄ and/or R₅ and R₆ may together form a substituted or unsubstituted heterocyclic group incorporating the attached nitrogen atom, and wherein one or more of R₁ to R₆ may comprise a substituent containing a monomer or polymer chain;

R₇ to R₁₅ which may be the same or different, are each independently selected from the group consisting of hydrogen atom, halogen atom, nitro group, ketone group, amide group, cyano group, carboxylate group, sulfonate group, substituted or unsubstituted aryl group, substituted or unsubstituted alkyl group and substituted or unsubstituted heterocyclic group, or pairs of substituents R₇ and R₈, R₁₀ and R₁₁ and/or R₁₃ and R₁₄ may together form a substituted or unsubstituted cyclic group, in which case each of R₉, R₁₂ and R₁₅ is as defined previously or is absent; and in which any substituent on any of groups R₇ to R₁₅ may contain a monomer or polymer chain; and

Ar₁₆ to Ar₁₈ are each selected from substituted and unsubstituted aryl groups, in which the substituent may contain a monomer or polymer chain.

The present invention also provides novel compounds within the range represented by formula (1a):

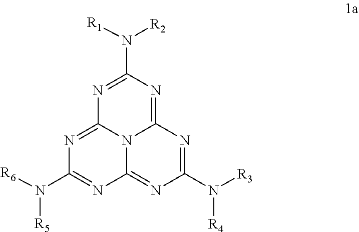

wherein:

R₁ to R₆, which may be the same or different, are each independently selected from the group consisting of substituted or unsubstituted aryl groups, substituted or unsubstituted heterocyclic groups, substituted or unsubstituted alkyl groups, and cyano, or each pair of substituents R₁ and R₂, R₃ and R₄ and/or R₅ and R₆ may together form a substituted or unsubstituted heterocyclic group incorporating the attached nitrogen atom, and wherein one or more of R₁ to R₆ may comprise a substituent containing a monomer or polymer chain.

The present invention also provides novel compounds within the range represented by formula (1b):

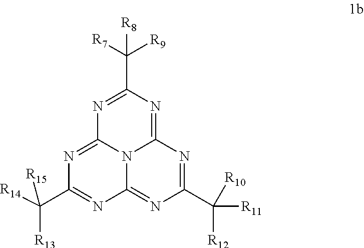

wherein:

R₇ to R₁₅ which may be the same or different, are each independently selected from the group consisting of hydrogen atom, halogen atom, nitro group, ketone group, amide group, cyano group, carboxylate group, sulfonate group, substituted or unsubstituted aryl group, substituted or unsubstituted alkyl group and substituted or unsubstituted heterocyclic group, or pairs of substituents R₇ and R₈, R₁₀ and R₁₁ and/or R₁₃ and R₁₄ may together form a substituted or unsubstituted cyclic group, in which case each of R₉, R₁₂ and R₁₅ is as defined previously or is absent; and in which any substituent on any of groups R₇ to R₁₅ may contain a monomer or polymer chain.

The present invention also provides novel compounds within the range represented by formula (1c):

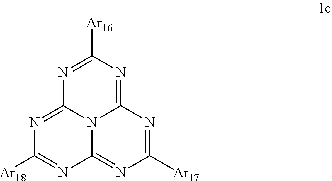

Ar₁₆ to Ar₁₈ are each selected from substituted and unsubstituted aryl groups, in which the substituent may contain a monomer or polymer chain.

The present invention also provides the use of a compound of formula (1) in an organic electroluminescent device.

### BRIEF DESCRIPTION OF THE DRAWINGS

FIG. 1 is a schematic illustration of the basic structure of an organic electroluminescent device according to a first embodiment of the invention.

FIG. 2 is a schematic illustration of the basic structure of an organic electroluminescent device according to a second embodiment of the invention.

FIG. 3 is a schematic illustration of the basic structure of an organic electroluminescent device according to a third embodiment of the invention.

FIG. 4 is a schematic illustration of the basic structure of an organic electroluminescent device according to a fourth embodiment of the invention.

FIG. 5 is a graph of voltage versus current density for compounds 2 and 5, and shows a characteristic of hole injection-ability for compounds 2 and 5.

FIG. 6 is a graph showing power efficiency comparison data between compound 2 of the present invention and prior art compound hexacyanohexaazatriphenylene (HAT) at low current.

### DETAILED DESCRIPTION OF THE PREFERRED EMBODIMENTS

The organic electroluminescent device according to the present invention is composed of organic compounds layer(s) aligned between an anode and a cathode.

The organic layer(s) may be constituted by:
a single layer doped with a compound of formula (1), or
multiple layers of which at least one layer may be doped with a compound of formula (1), or at least one layer is a layer comprised of a compound of formula (1) doped with a separate dopant, or
multiple layers of which at least one layer may be comprised entirely of a compound of formula (1).

The term “compound” is used in its broadest sense to refer to any chemical substance of formula (1), and includes polymers, monomers and the like. It will be understood that some forms of the compound of formula (1) are polymer forms.

The compounds of formula (1) may be classified into three subgroups, all sharing the same core ring structure. In the following we have described the features of each sub-group, although the definitions and described embodiments also apply to the compounds of general formula (1).

Formula (1a)

In formula (1a), one or more of R₁ to R₆ may be an aryl group, which may be unsubstituted, or substituted by a suitable substituent. The term “aryl” is well understood in the art of chemistry, and is used to refer to any aromatic substituent. The aromatic substituent preferably contains one or more rings, such as from one to four fused aromatic rings, and between 5 and 50 ring atoms. Aromatic substituents contain a set of covalently-bound atoms with:
a delocalized conjugated r system, most commonly an arrangement of alternating single and double bonds,
a coplanar structure, with all the contributing atoms in the same plane,
atoms of the system arranged in one or more rings, and
an even number of r delocalized electrons, but not a multiple of 4π electrons (thus 4n+2π electrons, where n=0 or a positive integer).

Any aromatic groups conforming to this rule are within the definition of aryl. The aryl group may be carbocyclic (i.e. contain carbon and hydrogen only) or may be heteroaromatic (i.e. contain carbon, hydrogen, and at least one heteroatom). The aryl group may be monocyclic such as a phenyl, or a polycyclic aryl group such as naphthyl or anthryl. Examples of aryl groups include a phenyl group, biphenyl group, terphenyl group, naphthyl group, anthryl group, pyrenyl group, etc.

In formula (1a), one or more of R₁ to R₆ may be an alkyl group, which may be unsubstituted, or substituted by a suitable substituent. The alkyl group for the formula (1a) may be a linear or branched alkyl group or cyclic alkyl group, comprising of between (and including) 1 and 20 carbon atoms. Examples of linear alkyl groups include methyl, propyl or decyl, and examples of branched alkyl groups include iso-butyl, tert-butyl or 3-methyl-hexyl. Examples of cyclic alkyl groups include mono cyclohexyl and fused alkyl cyclic ring systems.

In formula (1a), one or more of R₁ to R₆ may be a cyano group.

In formula (1a), one or more of R₁ to R₆ may be a heterocyclic group, which may be unsubstituted, or substituted by a suitable substituent. The term “heterocyclic”, and similarly “heterocyclic group” or “heterocyclic ring” is well understood in the art of chemistry, and is used to refer to any cyclic groups containing one or more rings, such as between one and four rings, and between 5 to 50 (preferably 5 to 20) ring atoms, of which at least one atom is a heteroatom. The heteroatoms may be selected from one or more of O, N, S and Si. The heterocyclic group for the formula (1) may be a 5 or 6 membered heterocyclic ring comprising carbon atoms with one or more of any of the following atoms: nitrogen, oxygen, sulphur and silicon, such as pyrrolyl, thienyl, pyridyl or pyridazinyl. The heterocyclic group may comprise a single heterocyclic ring, or more than one linked or fused rings, with at least one ring containing a heteroatom. One subclass of heterocyclic groups are the heteroaromatic (or heteroaryl) groups, which are aromatic groups containing one or more heteroatoms selected from one or more of O, N and S. Such heteroaromatic groups also fall within the definition of aryl group. Some specific examples for heterocyclic groups are pyrrole, triazole, imidazole, pyrazole, 1,2,5-oxathiazole, isoxazole, oxazole, furan, pyran, pyrone, thiazole, isothiazole, pyrrolidine, pyrroline, imidazolidine, pyrazolidine. Other examples include moieties of benzimidazole, thiophene, benzothiophene, oxadiazoline, indoline, carbazole, pyridine, quinoline, isoquinoline, benzoquinone, pyrazoline, imidazolidine, piperidine, etc.

According to some embodiments, each pair of substituents R₁ and R₂, R₃ and R₄, and/or R₅ and R₆ may together form a substituted or unsubstituted heterocyclic group incorporating the attached nitrogen atom. In other words, the segment of the compound of formula (1a) represented by —NR₁R₂ or —NR₃R₄ or —NR₅R₆ may form a substituted or unsubstituted heterocyclic group. The term heterocyclic group has been defined previously. Examples of suitable groups for —NR₁R₂ (or —NR₃R₄ or —NR₅R₆) include a pyrrolyl group(-(NC₃H₄)), 1-indolyl group, 1-pyrazolidinyl group, 9-carbazole group or 10-phenothiazinyl group. The heterocyclic group may be monocyclic or polycyclic. According to some embodiments, the heterocyclic group is polycyclic. An example of a polycyclic heterocyclic group within this class is carbazole. Some groups within this class, such as the carbazole group, may be considered to correspond to the situation where R₁ and R₂ (or R₃ and R₄, or R₅ and R₆) each comprise substituted or unsubstituted aryl or heteroaryl groups, and the two groups R₁ and R₂ are connected to each other to form a fused ring structure. In the case of carbazole, the groups R₁ and R₂ are aryl groups (specifically phenyl groups) and are connected to each other through a direct bond between two ring atoms to form a fused ring structure. The groups R₁ and R₂ (or R₃ and R₄, or R₅ and R₆) could alternatively be connected to each other through any divalent linking group or fused ring structure.

In formula (1a), the aryl or alkyl or heterocyclic group may additionally have one or more substituents selected from any suitable substituents known in the art. Suitable substituents may be selected from the group consisting of: halogen atom, nitro group, ketone group, amide group, cyano group, carboxylate group, sulfonate group, alkoxy group, aryloxy group, amino group, alkylamino group, arylamino group, a monomer or polymer chain and another aryl, alkyl or heterocyclic group, in which each of the aryl, alkyl or hetercyclic groups may be further substituted by one or more further substituents. Thus, the further substituents on the aryl, alkyl or heterocyclic group substituents may be selected from one or more of a halogen atom, nitro group, ketone group, amide group, cyano group, carboxylate group, sulfonate group, alkoxy group, aryloxy group, amino group, alkylamino group and arylamino group.

The substituents may be linked to the aryl, alkyl or heterocyclic group directly by one atom or fused by more than one atom or via a heteroatom such as nitrogen, oxygen, sulphur and silicon. In some cases the substituent may be linked by two or more points of attachment, as in the case of a divalent alkyl group, or an ethylenedioxy group (i.e. —O—CH₂CH₂—O—).

The term “halogen” or halo refers to fluorine, chlorine, bromine, etc. Nitro refers to —NO₂. Ketone refers to compounds containing the group —C(═O)-alkyl or —C(═O)-aryl, where alkyl and aryl are as defined previously. Acetyl (—C(═O)—CH₃ is one specific example.

The term amide refers to substituents containing the group —C(O)NR′R″, wherein R and R″ are selected from H, alkyl, aryl or alkyl-aryl groups, which have been defined previously. The term “imide” refers to substituents containing the group —C(O)NR′C(O)R″, wherein R and R″ are selected from H, alkyl, aryl or alkyl-aryl groups. The term “imine” refers to substituents containing the group —C(═NR′)R″, wherein R and R″ are selected from H, alkyl, aryl or alkyl-aryl groups. The term “amidine” refers to substituents containing the group —C(═NR′)NR″R⁻, wherein R′, R″ and R⁻ are selected from H, alkyl, aryl or alkyl-aryl 2 0 groups.

Cyano refers to —C≡N. Hydroxyl refers to —OH. Carboxylate refers to the carboxylate anion —CO₂R⁻, and encompasses carboxylic acids, esters and salts thereof Sulfonate group refers to sulfonic acids, esters and salts thereof. Alkoxy refers to the group —O-alkyl, where alkyl is as defined previously. Aryloxy refers to the group —O-aryl, where aryl is as defined previously.

The term “amino” refers to the amino group —NH₂. The term alkylamino refers to secondary and tertiary alkylamino groups containing one or two alkyl groups on the nitrogen atom. Examples of an “alkylamino group” include dimethylamino group, diethylamino group, dihexylamino group, etc. The term arylamino refers to secondary and tertiary arylamino groups containing one or two aryl groups on the nitrogen atom. Examples of an “arylamino group” include a diphenylamino group, ditolylamino group, isopropyldiphenylamino group, t-butyldiphenylamino group, diisopropyldiphenylamino group, di-t-butyldiphenylamino group, dinaphthylamino group, naphthylphenylamino group, etc.

According to one class of compounds of formula (1a), the compound may contain a monomer or polymer chain. The inclusion of monomer or polymer chains in the compounds of formula (1) (including (1a), (1b) and (1c)) is described in further detail below following discussion of the compounds of formulae (1b) and (1c).

Formula 1b

In formula (1b), one or more of R₇ to R₁₅ may be selected from the group consisting of a hydrogen atom, halogen atom, nitro group, ketone group, amide group, cyano group, carboxylate group, sulfonate group, substituted or unsubstituted aryl group, substituted or unsubstituted alkyl group and substituted or unsubstituted heterocyclic group. These terms have been defined above in the context of Formula (1a), and those definitions apply equally here.

The optional substituents on the alkyl, aryl and heterocyclic groups are the same as those described above for formula (1a), and include halogen atom, nitro group, ketone group, amide group, cyano group, carboxylate group, sulfonate group, alkoxy group, aryloxy group, amino group, alkylamino group, arylamino group, a monomer or polymer chain and another aryl, alkyl or heterocyclic group, in which each of the aryl, alkyl or hetercyclic groups may be further substituted by one or more further substituents. Again, the further substituents on the aryl, alkyl or heterocyclic group substituents may be selected from one or more of a halogen atom, nitro group, ketone group, amide group, cyano group, carboxylate group, sulfonate group, alkoxy group, aryloxy group, amino group, alkylamino group and arylamino group.

The substituents may be linked to the aryl, alkyl or heterocyclic group directly by one atom or fused by more than one atom or via a heteroatom. In some cases the substituent may be linked by two or more points of attachment, as in the case of a divalent alkyl group, or an ethylenedioxy group (i.e. —O—CH₂CH₂—O—).

In another embodiment, pairs of substituents R₇ and R₈, R₁₀ and R₁₁ and/or R₁₃ and R₁₄ may together form a substituted or unsubstituted cyclic group, in which case each of R₉, R₁₂ and R₁₅ is as defined previously or is absent; and in which any substituent on any of groups R₇ to R₁₅ may contain a monomer or polymer chain. The term “cyclic group” is used in its broadest sense to refer to cyclic rings and linked or fused ring systems, which may be carbocyclic or heterocyclic, and may be aliphatic, aromatic, saturated or unsaturated. The ring may be carbocyclic groups (in which all of the ring atoms are carbon atoms, such as cyclohexyl), heterocyclic groups (as described previously), and aromatic or aryl groups (which may be carbon-based aromatic groups or heteroaromatic groups). The cyclic group may contain a single ring, or up to 3 linked or fused rings.

According to one class of compounds of formula (1b), at least one of R₇, R₈ and R₉, at least one of R₁₀, R₁₁ and R₁₂, and at least one of R₁₃, R₁₄ and R₁₅, is not a hydrogen atom.

According to one class of compounds of formula (1b), the compound may contain a monomer or polymer chain. This aspect is described in further detail below.

Formula (1c)

In formula (1c), Ar₁₆ to Ar₁₈ are each selected from substituted and unsubstituted aryl groups. The term “aryl” has been defined previously.

The aryl group may be substituted or unsubstituted. The substituents are as described previously for aryl groups in the context of formulae (1a) and (1b).

Monomer or Polymer Chain Substituents

According to one class of compounds of formula (1), (and similarly of formulae (1a) to (1c)), the compound may contain a monomer or polymer chain. The compound represented as formula (1) may be connected to a monomer or polymer chain through at least one of the groups of R₁ to R₆ (in the case of formula (1a)), or through at least one of the groups R₇ to R₁₅ (in the case of formula (1b)), or through a substituent on one or more of Ar₁₆ to Ar₁₈ (in the case of formula (1c)).

The monomer or polymer chain may be attached via any suitable divalent linking group, which may be referred to as —R₁₉—. Examples of divalent linking groups include a direct bond, —O—, —NH—, —Nalkyl-, —Naryl-, -alkyl- (such as —(CH₂)ₓ—), —CO₂—, —CO—, -aryl-, -heteroaryl-, and combinations thereof. Combinations include, as examples, —O-aryl-, —NH-aryl-, —Nalkyl-aryl-, —(CH₂)ₙ-aryl-. Thus, one of the substituents on R₁ to R₆ may be —R₁₉-monomer or —R₁₉-polymer.

The term “monomer” refers to any monomer unit that can be polymerised. Suitable monomers include —′CR₂₀═CHR₂₁, where R₂₀ is hydrogen, alkyl, aryl or heteroaryl, and R₂₁ is hydrogen, a halogen atom, nitro group, ketone group, acrylate group, amide group, cyano group, carboxylate group, sulfonate group, an aryl, an alkyl or a heterocyclic group, or R₂₀ and R₂₁ together form a cyclic or heterocyclic group, which may be substituted or substituted. Each of these substituents may be further substituted by one or more further substituents selected from the range of possible substituents identified previously. The point of attachment of the monomer to the remainder of the compound may be via the carbon atom marked ′C. In the case where R₂₀ and R₂₁ form a cyclic or heterocyclic group, the point of attachment may be via an atom in the cyclic or heterocyclic group, and in this case, ′C contains a further substitutent R₂₂ which is selected from hydrogen, alkyl, aryl and heteroaryl. The monomer may be polymerised subsequently to form a polymeric version of the compound of formula (1).

The term “polymer” or “polymer chain” refers to any polymer or polymer segment. Examples of the compounds of the present invention represented below containing polymers/polymer segments illustrate some examples. Examples include —(′CH—CH₂)ₙ— and —(′CR₂₀—CHR₂₁)ₙ—, where R₂₀ is selected from hydrogen, alkyl, aryl and heteroaryl, and R₂₁ is selected from hydrogen or any suitable substituent, such as a halogen atom, nitro group, ketone group, acrylate group, amide group, cyano group, carboxylate group, sulfonate group, an aryl, an alkyl or a heterocyclic group, or R₂₀ and R₂₁ together form a cyclic or heterocyclic group, which may be substituted or unsubstituted. When R₂₀ and R₂₁ together form a cyclic or heterocyclic group, the cyclic or heterocyclic group includes the attached carbon atoms. Each of these substituents may be further substituted by one or more further substituents selected from the range of possible substituents identified previously. The point of attachment of the polymer or polymer segment may be via the carbon atom marked ′C. In the case where R₂₀ and R₂₁ form a cyclic or heterocyclic group, the point of attachment of the polymer or polymer segment may be via an atom in the cyclic or heterocyclic group, and in this case, ′C contains a further substitutent R₂₂ which is selected from hydrogen, alkyl, aryl and heteroaryl. As described above, the polymer or polymer segment is attached to the compound via any divalent linking group or combination of groups.

The term “heterocyclic group” in the context of monomers and polymers has the same meaning provided for the term previously. The term “cyclic group” has also been defined previously.

Compounds of formula (1) which contain a polymer chain substituent may be formed through any suitable technique, such as RAFT polymerisation, or another form of living polymerisation. RAFT and living polymerisation techniques are known in the field of polymers, and details can be obtained from publications on RAFT and living polymerisation techniques. Also noted are patent applications filed in the name of the present applicant in the field of RAFT polymerisation, the entirety of which publications are incorporated by reference. Compounds of formula (1) containing a monomer unit can be polymerised through any suitable technique as described above to form a polymeric form of the compound of formula (1).

Specific examples of the compound represented as formula (1) may include example compounds No. (2) to (61) shown below but are, however, not restricted to those compounds.

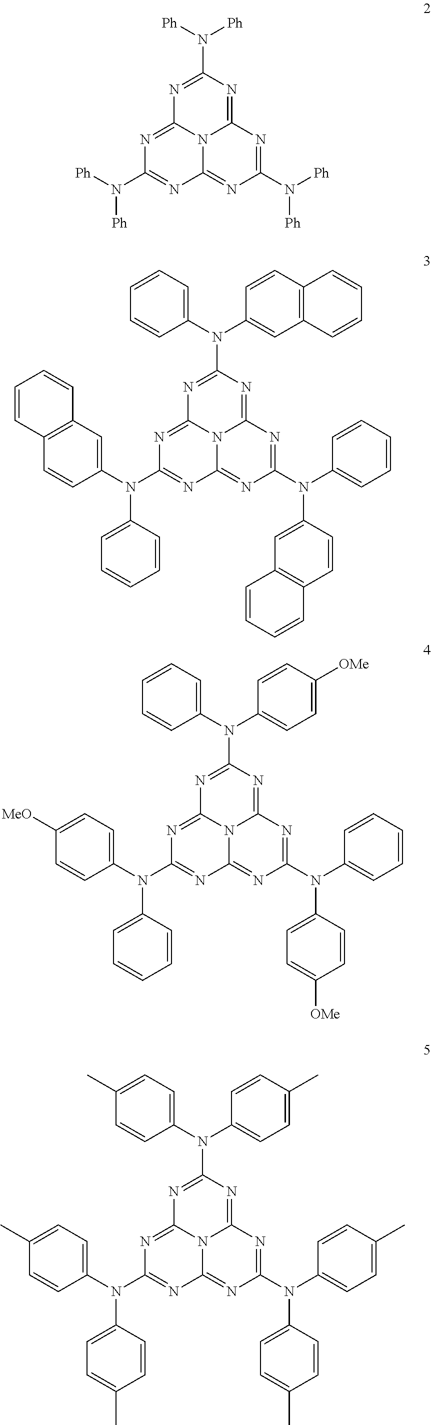

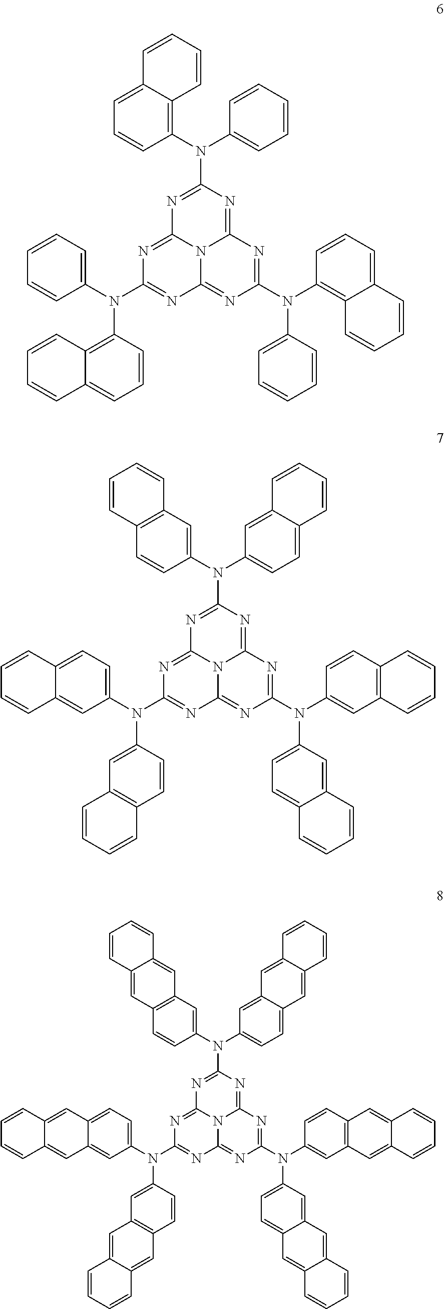

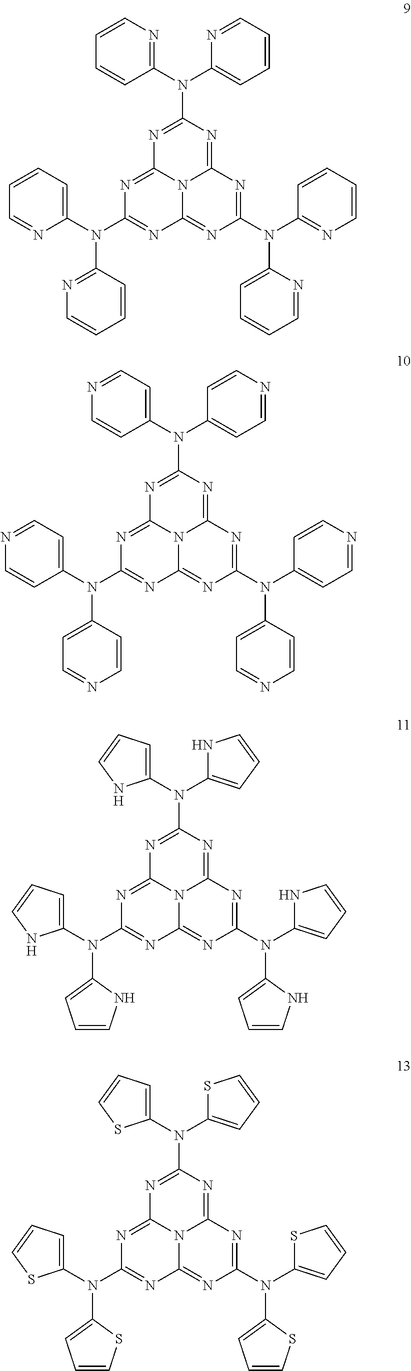

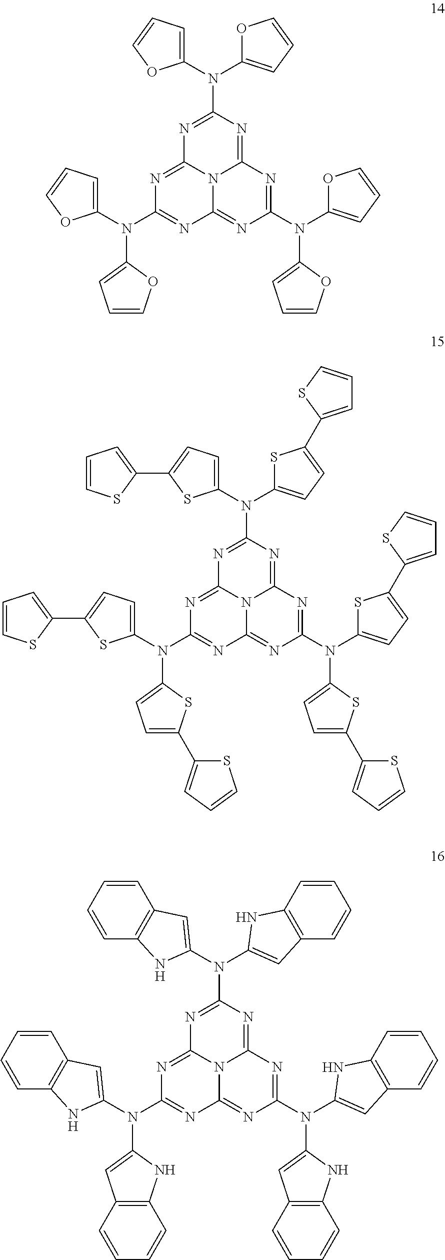

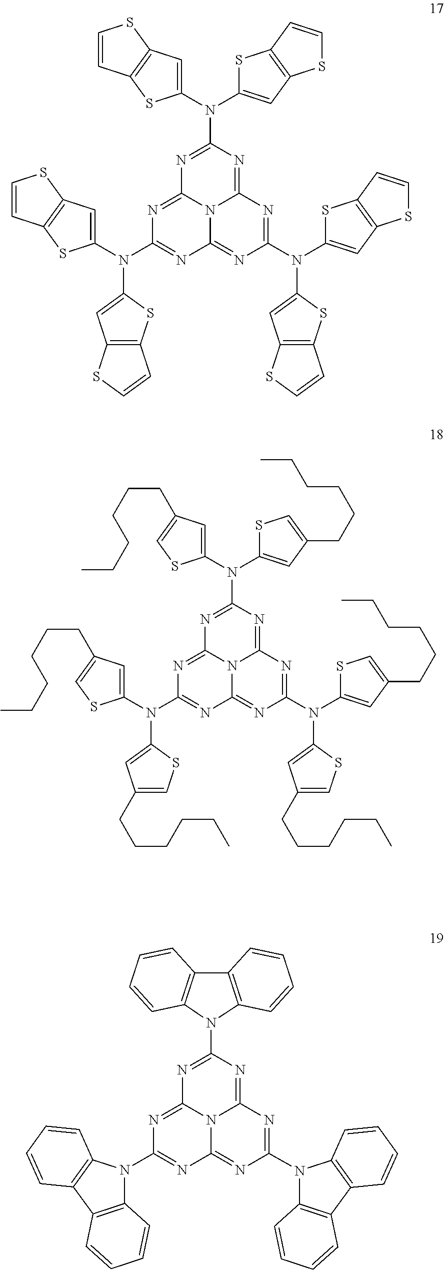

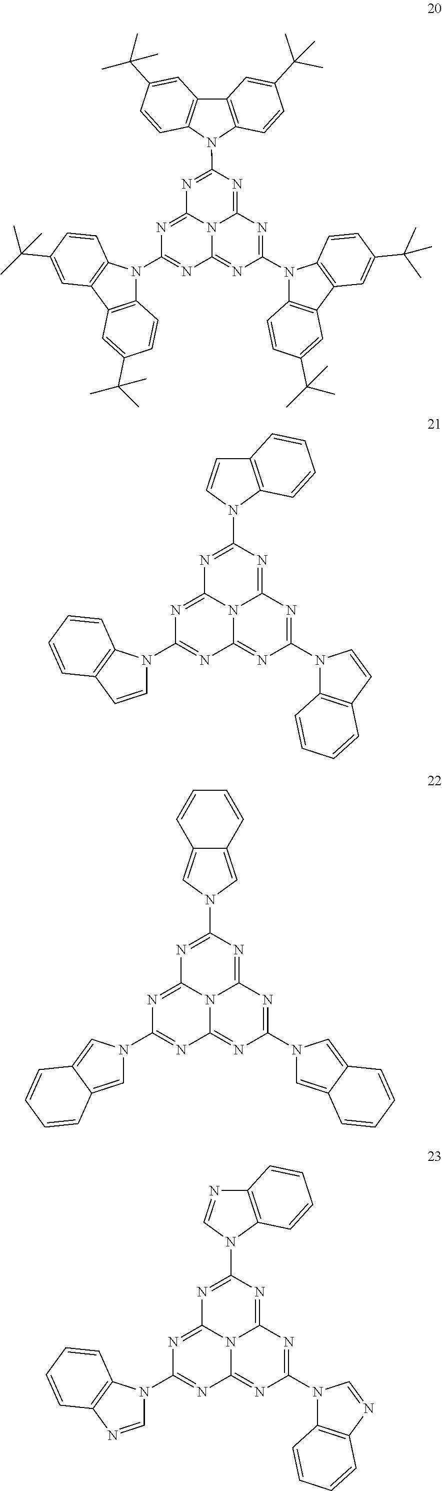

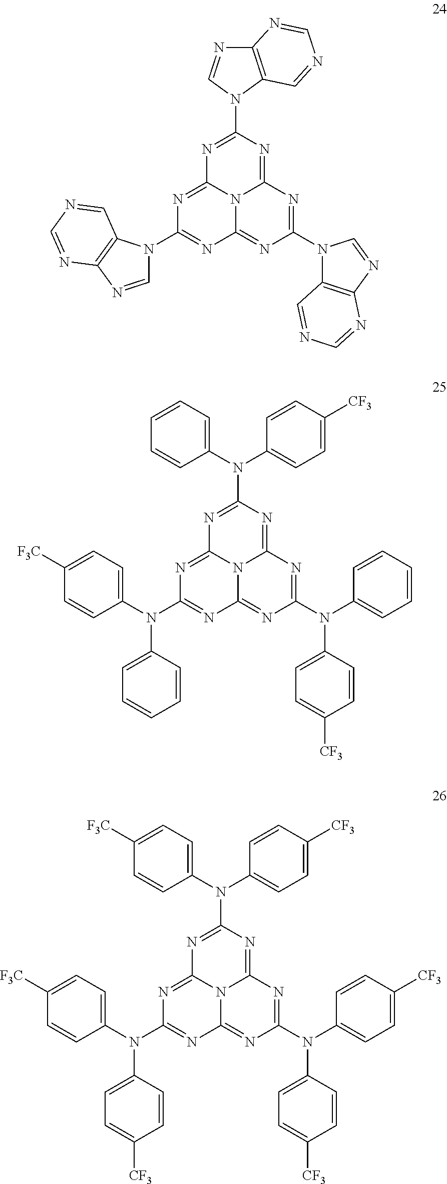

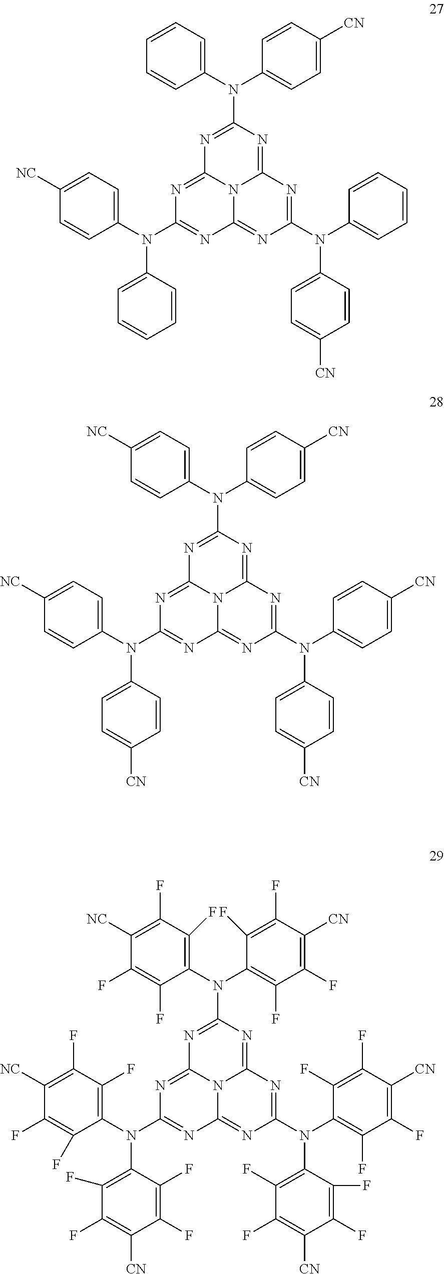

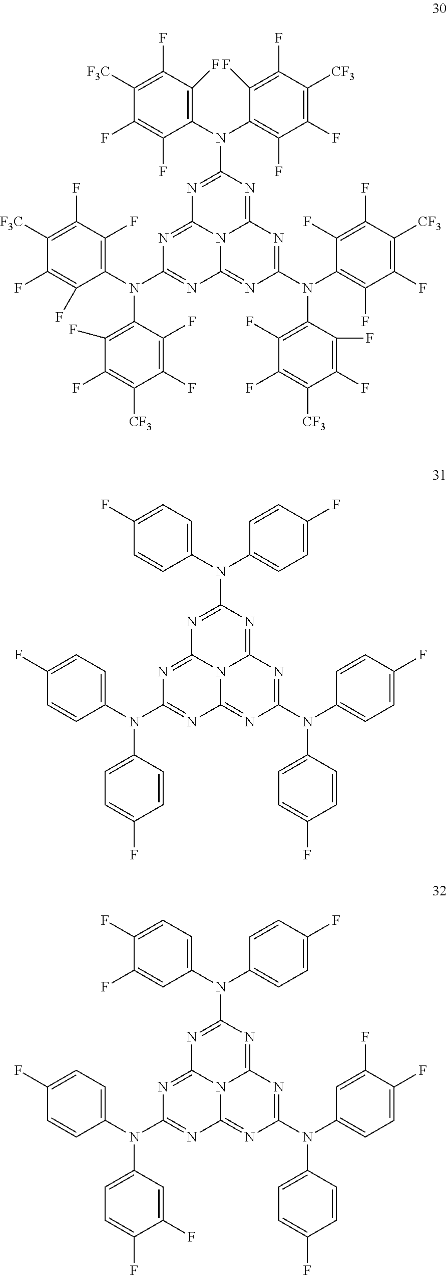

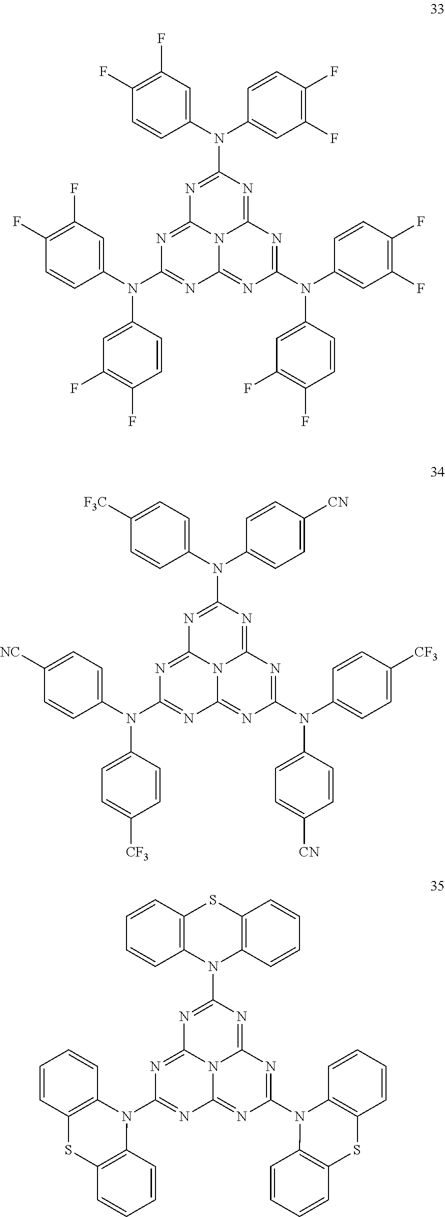

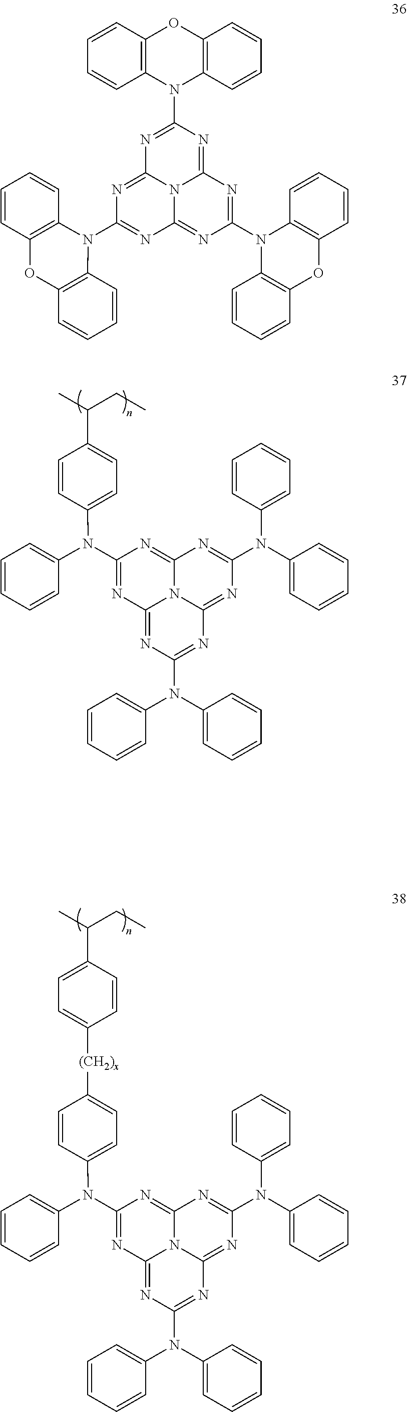

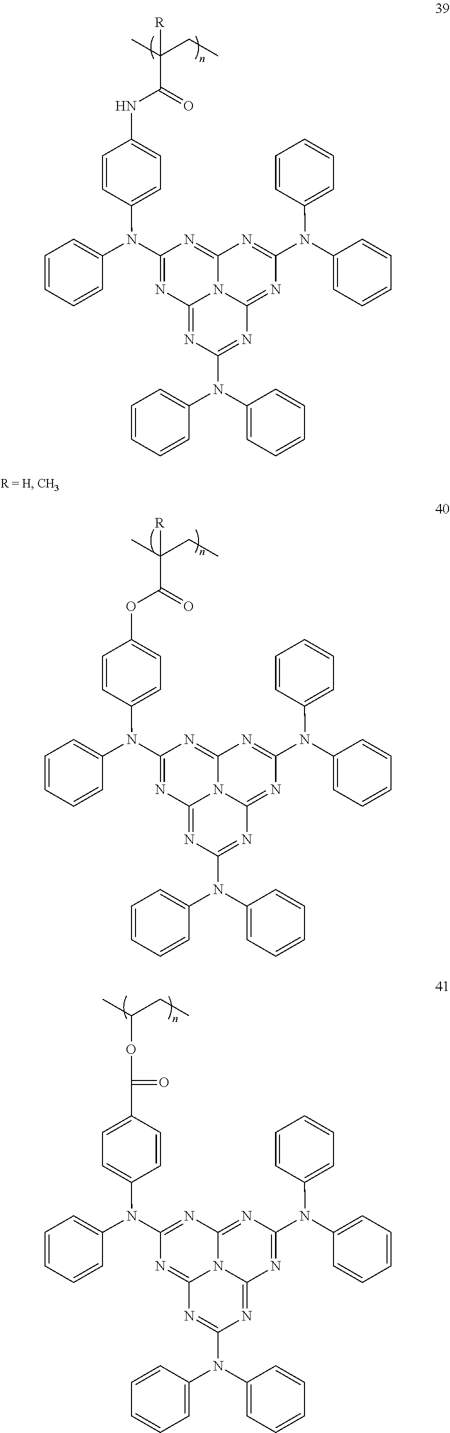

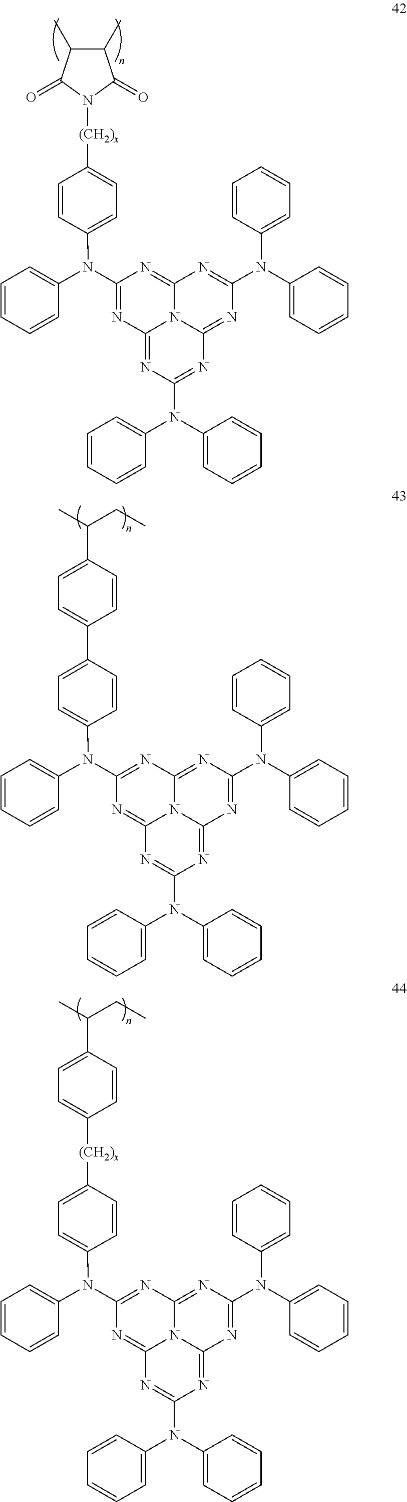

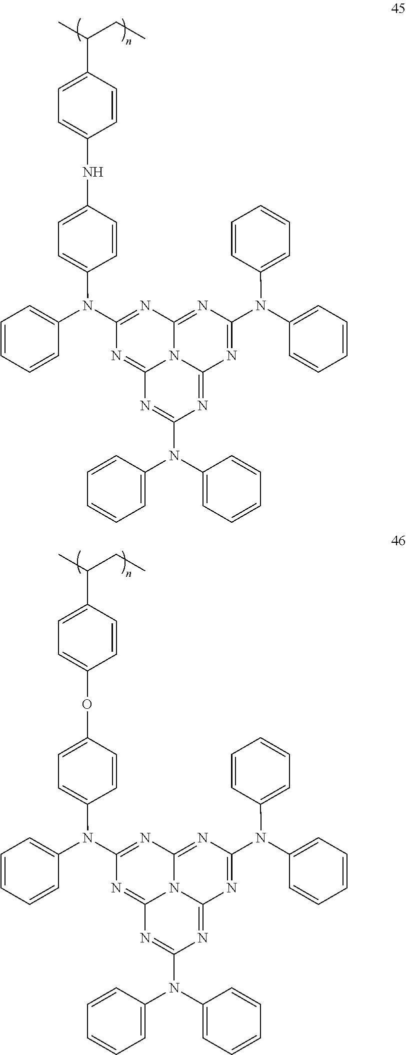

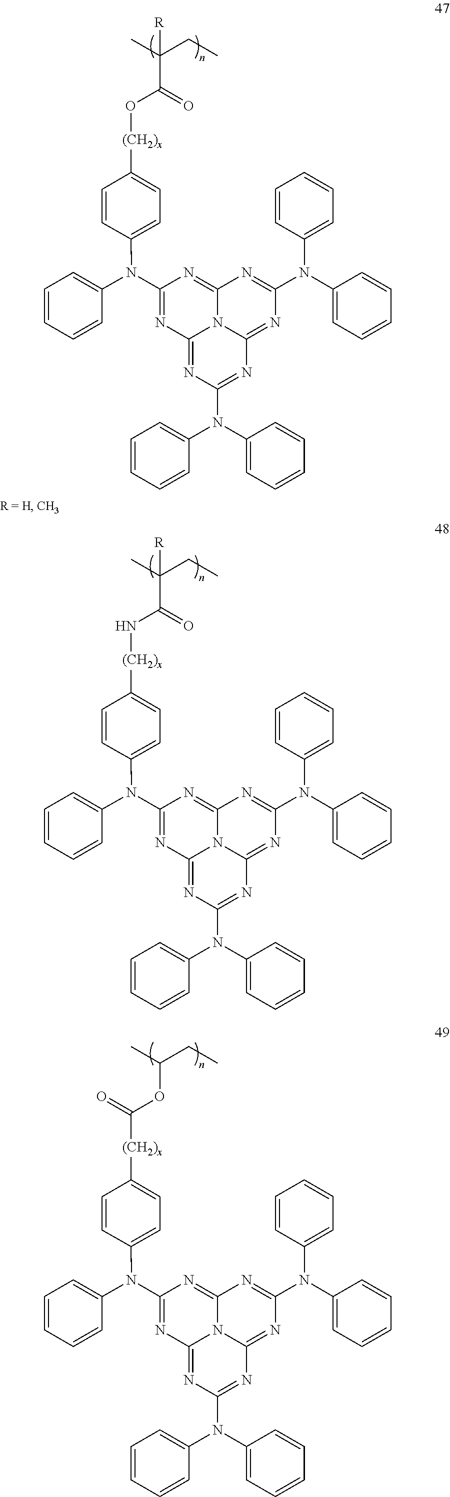

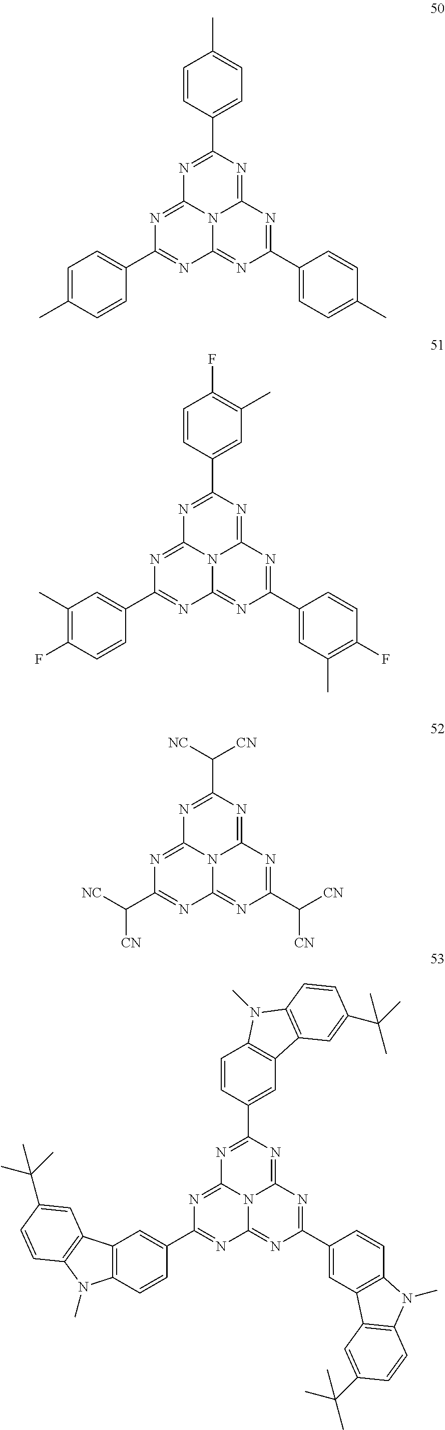

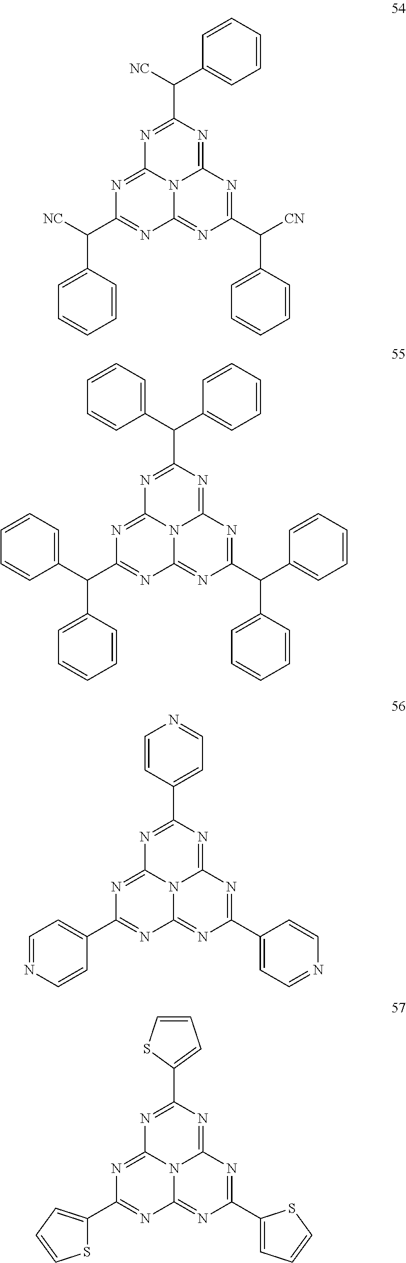

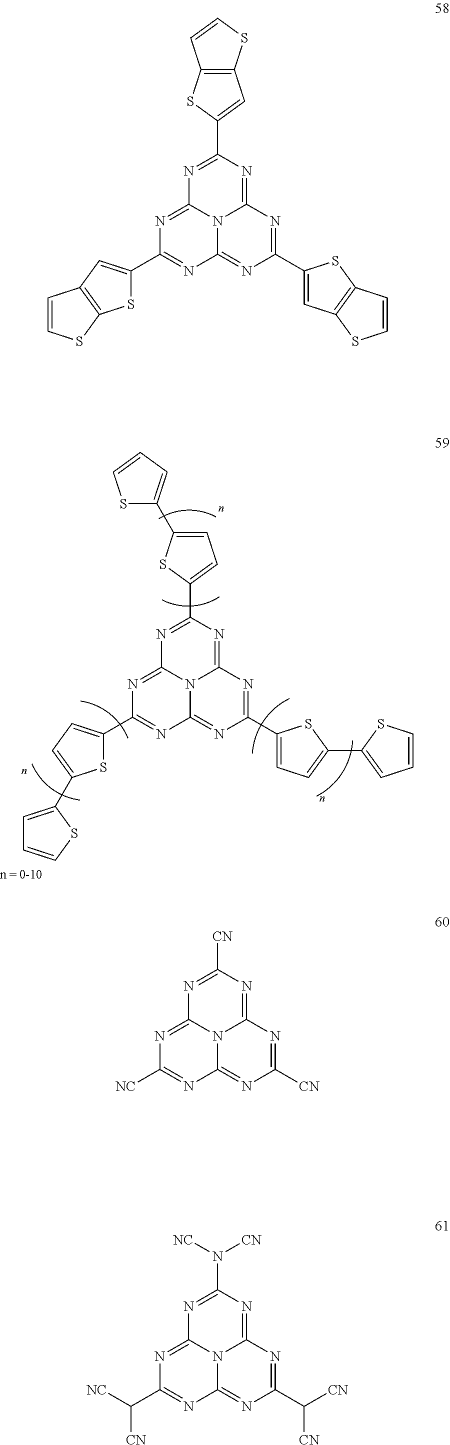

As described above, the organic layer(s) may be constituted by:
a single layer doped with a compound of formula (1), or
multiple layers of which at least one layer may be doped with a compound of formula (1) or at least one layer is a layer comprised of a compound of formula (1) doped with a separate dopant, or
multiple layers of which at least one layer may be comprised entirely of a compound of formula (1).

In the organic luminescence device of the present application, the organic compound layer comprising the above-mentioned compound of the formula (1) may be formed separately, or together, with the other layers (if any other layers are present) between the pair of electrodes (cathode and anode). Suitable formation techniques include vacuum deposition or solution process.

The thickness of the organic compound layer may be preferably less than at most 10 μm, more preferably less than 0.5 μm, even more preferably 0.001-0.5 μm.

Specific embodiments of the invention will now be described in further detail with reference to the accompanying figures, which illustrate a range of possible arrangements for the device of the present invention. It will be understood that these embodiments are provided by way of example only, and are not intended to limit the scope of the invention.

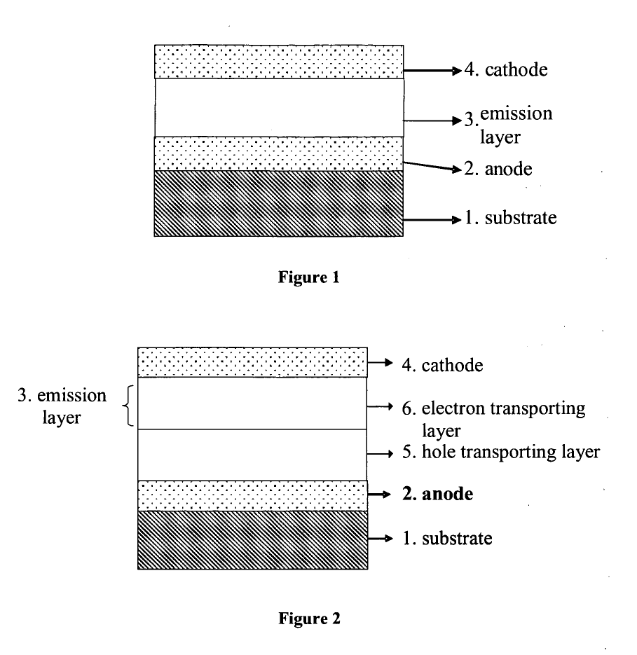

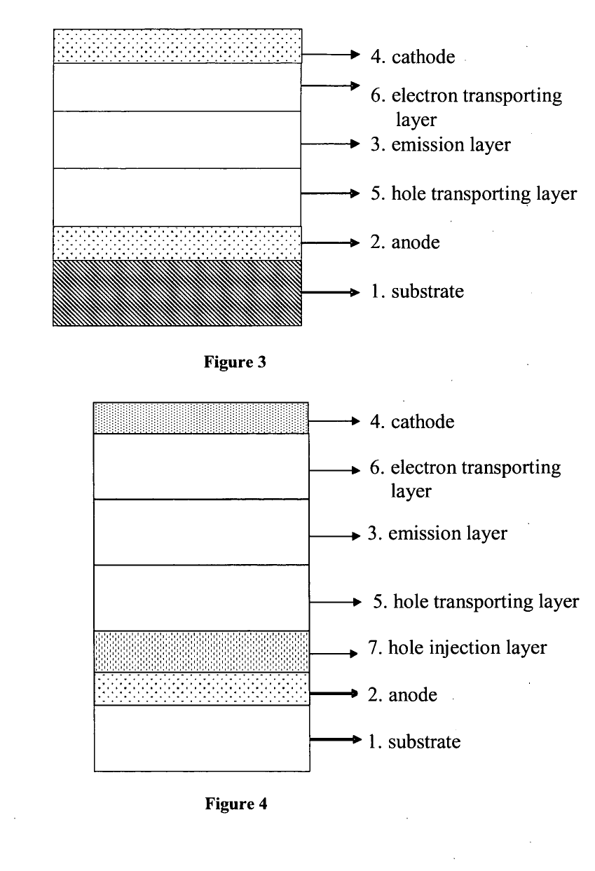

The electroluminescent device of embodiments of the present application may have a single layer structure comprised of only compound as defined by formula (1) as shown in FIG. 1 or be a multiple layered structure of two or more layers as shown in FIGS. 2 and 3.

More specifically, FIG. 1 is a schematic cross section of a first embodiment of the organic electroluminescent device of the present invention. In FIG. 1, the organic electroluminescent device includes a substrate 1, an anode 2 (deposited on the substrate 1), an emission layer 3 (deposited on the anode 2) and a cathode 4 (deposited on the emission layer 3). In this embodiment, the emission layer 3 forms a single organic compound type-layer. This single layer may be composed entirely of a compound having hole transporting ability, electron transporting ability and luminescence ability (associated with the re-combination of electrons and holes) based on its own properties, or through combination with a dopant that enhances the performances of the hole transporting ability, the electron transporting ability and luminescence ability of host compound. According to some embodiments, the compound of formula (1) can serve as a hole transporting layer with a dopant. According to other embodiments, the compound of formula (1) can function as a dopant. According to other embodiments, the compound of formula (1) can serve as a separate hole injection layer, as described further below.

In FIG. 1, the emission layer 3 may preferably have a thickness of 5 nm to 1 μm, more preferably 5 to 50 nm.

FIG. 2 shows another embodiment of the organic electroluminescent device of the present invention in the form of a multiple layer-type device comprised of a hole transporting layer 5 and an electron transporting layer 6.

Referring to FIG. 2, the organic luminescent device includes a substrate 1, an anode 2 (deposited on the substrate 1), the hole transporting layer 5 (deposited on the anode 2), the electron transporting layer 6 (deposited on the hole transporting layer 5) and a cathode (deposited on the electron transporting layer 6). In this embodiment, either one or both of the hole transporting layer 5 and the electron transporting layer 6 may contain an emissive compound as dopant(s) for forming an emission layer 3. In this case, the hole transporting layer 5 and the electron transporting layer 6 may be comprised of non-luminescent compound(s), respectively. The compound of formula 1 can form the hole transporting layer 5, or a component of the hole transporting layer.

In the embodiment of FIG. 2, each of the hole transporting layer 5 and the electron transporting layer 6 may have the thickness of 5 nm to 1 μm, more preferably 5 nm to 50 nm.

FIG. 3 shows another embodiment of the organic electroluminescent device of the present invention in the form of a multiple layer-type device comprising a hole transporting layer 5, an emission layer 3, an electron transporting layer 6. In FIG. 3, the organic luminescent device includes a substrate 1, an anode 2 (deposited on the substrate 1), the hole transporting layer 5 (deposited on the anode 2), the emission layer 3 (deposited on the hole transporting layer 5), the electron transporting layer 6 (deposited on the emission layer 3) and a cathode (deposited on the electron transporting layer 6). In this embodiment, each of the hole transporting layer, the emission layer and the electron transporting layer may be formed by use of a hole transporting compound, an emissive compound and an electron transporting compound, respectively or as a mixture of these kinds of compounds. The compound of formula 1 can form the hole transporting layer 5, or a component of the hole transporting layer.

FIG. 4 shows another embodiment of the organic electroluminescent device of the present invention with multiple layers comprising a hole injection layer 7, a hole transporting layer 5, an emission layer 3 and an electron transporting layer 6. In FIG. 4, the organic luminescent device includes a substrate 1, an anode 2 (deposited on the substrate 1), the hole injection layer 7 (deposited on the anode 2), the hole transporting layer 5 (deposited on the hole injection layer), the emission layer 3 (deposited on the hole transporting layer 5), the electron transporting layer 6 (deposited on the emission layer 3) and a cathode (deposited on the electron transporting layer 6). In this embodiment, each of the hole injection layer, the hole transporting layer, the emission layer and the electron transporting layer may be formed by use of a hole injection compound, a hole transporting compound, an emissive compound and an electron transporting compound, respectively or as a mixture of these kinds of compounds. The compound of formula 1 can form the hole injection layer 7 and/or the hole transporting layer 5 (or a component thereof).

In FIGS. 1, 2, 3 and 4, each layer of 3, 5, 6, and 7 may be formed by either vacuum deposition or wet process using low molecule or polymer compound or mixture of low molecule and polymer compound. Each thickness of the layer 3, 5 and 6 may preferably be ranging from 1 nm to 1 μm. Each of the thickness of the cathode and the anode may be preferably 100-200 nm.

The organic layer structures in the devices shown in FIGS. 1, 2, 3 and 4 represent the basic structure, respectively, so that the structure may be appropriately optimized depending on characteristics demanded. Examples of suitable modifications include the incorporation of one or more additional layers.

For example, the hole transporting layer may be altered to comprise a hole injection layer (deposited on the anode) and hole transporting layer (deposited on the hole injection layer).

More specific embodiments of the device structure other than those of FIGS. 1, 2, and 4 are shown below, but not restricted to these device structures.
(1) Anode/hole transporting layer/emission layer/electron transporting layer/electron injection layer/cathode
(2) Anode/hole injection layer/emission layer/electron transporting layer/electron injection layer/cathode
(3) Anode/insulating layer/hole transporting layer/emission layer/electron transporting layer/cathode
(4) Anode/hole transporting layer/emission layer/electron transporting layer/insulating layer/cathode
(5) Anode/inorganic semiconductor/insulator/hole transporting layer/emission layer/insulator/cathode
(6) Anode/insulating layer/hole transporting layer/emission layer/electron transporting layer/insulating layer/cathode
(7) Anode/insulating layer/hole injection layer/hole transporting layer/emission layer/electron transporting layer/electron injection layer/cathode
(8) Anode/insulating layer/hole injection layer/hole transporting layer/emission layer/electron transporting layer/electron injection layer/insulating layer/cathode

In the embodiments described above, more preferable device structures are (1), (2), (3), (7) and (8), although this is not a restriction. According to some embodiments, the compound of the formula (1) may be formed as a hole injection layer or a hole generation layer. In this case, the hole injection layer or the hole generation layer has a thickness of 1 nm to 1 μm, more preferably 1-50 nm. According to some embodiments, there is provided the use of the compound of formula (1) as a hole injection material, or a hole generation material, as a hole injection layer or a hole generation layer, or as a dopant in a hole transporting layer.

In some embodiments, the compound of the formula (1) may be used in combination with a hole transporting compound (or material), an electron transporting compound and/or an emission compound, examples of which may include the following.

Hole Transporting Materials/Compounds:

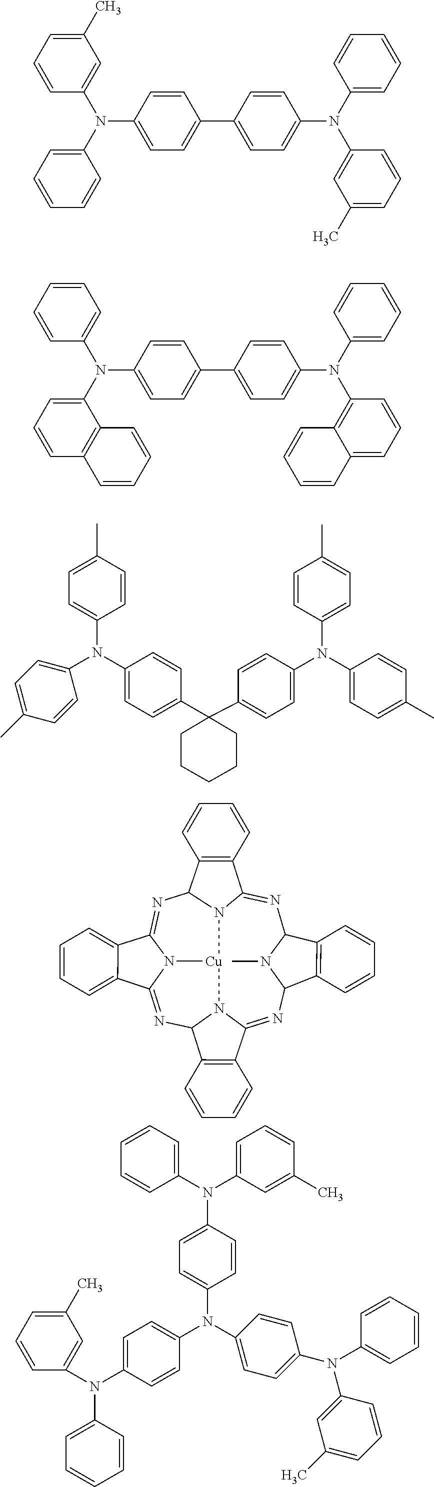

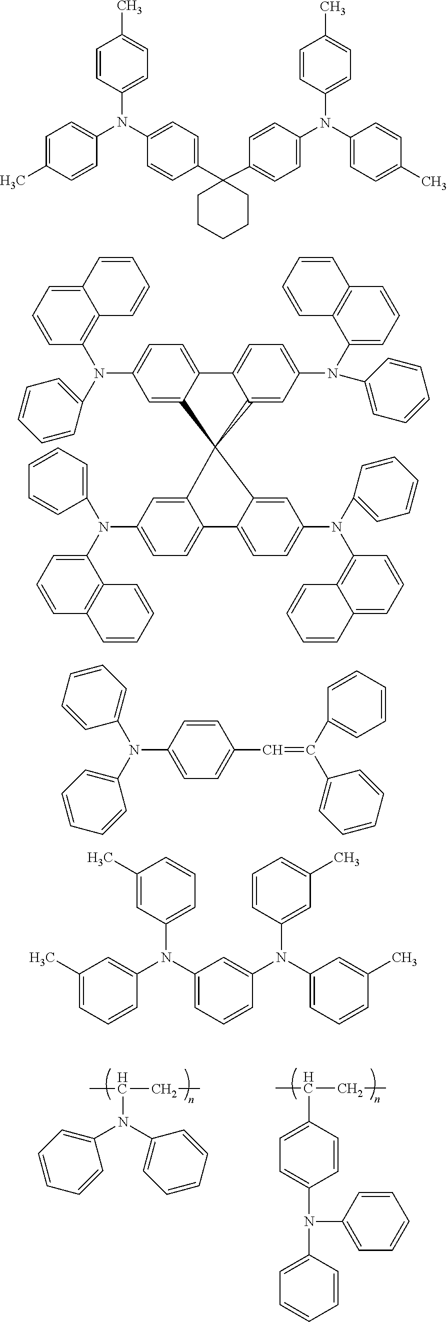

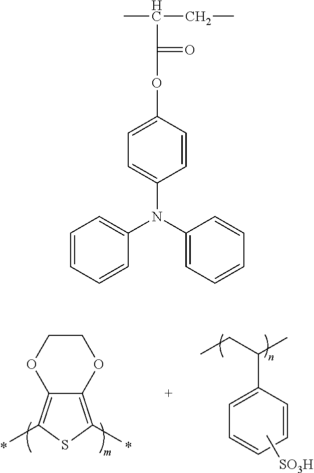

Electron Transporting Materials/Compounds:

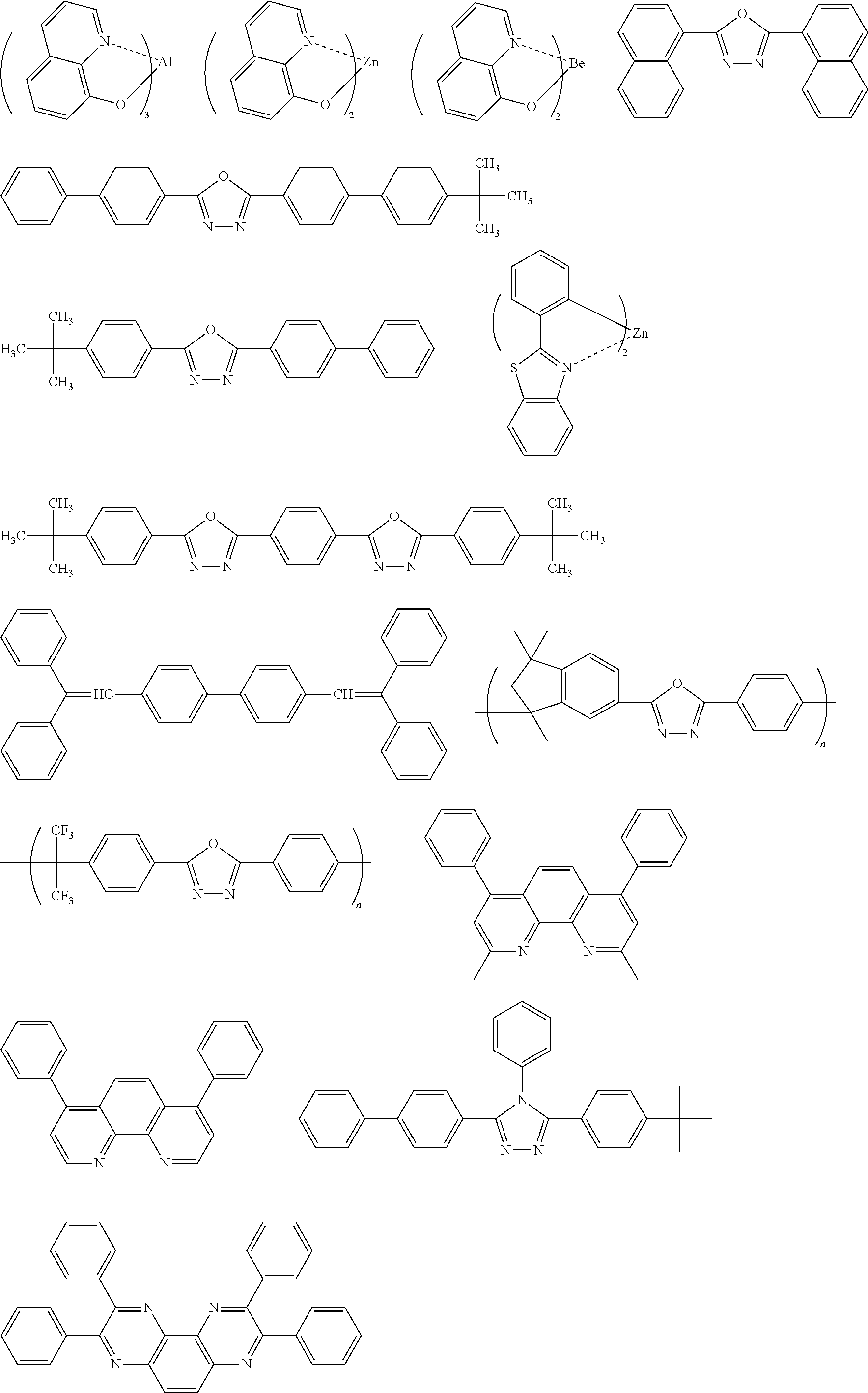

Emission Materials/Compounds:

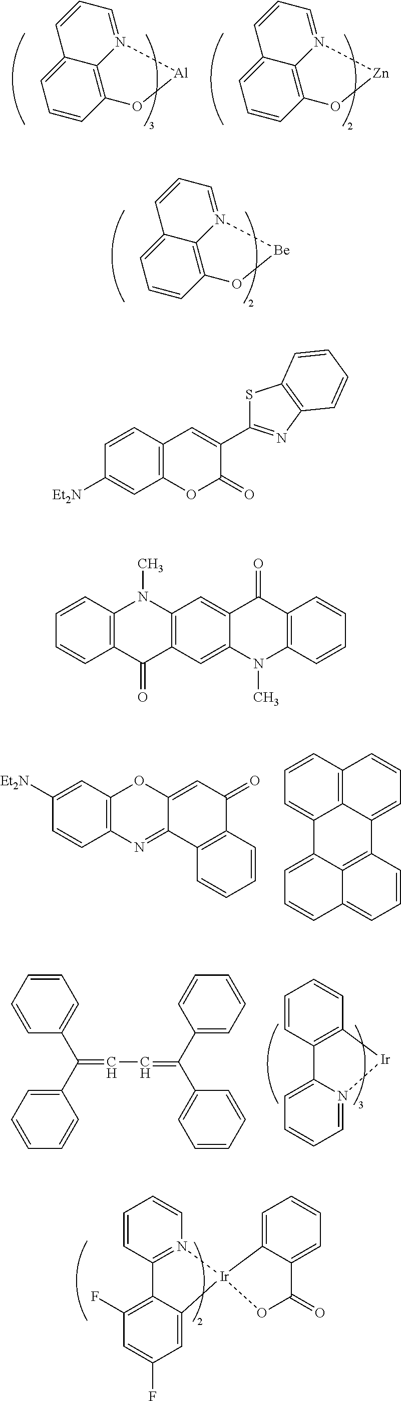

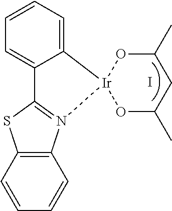

As a material for the anode (e.g. 2 in FIGS. 1-4), it is preferred to use one having a large work function, examples of which may include metals, such as gold, platinum, nickel, palladium, cobalt, selenium, vanadium and their alloys; metal oxides, such as tin oxide, zinc oxide, indium zinc oxide (IZO) and indium tin oxide (ITO) and electroconductive polymers, such as polyaniline, polypyrole and polythiophene and derivatives thereof. These compounds may be used singly or in combination of two or more species.

As a material for the cathode (e.g. 4 in FIGS. 1-4), it is preferred to use one having a smaller work function, usually under 4.0 eV, examples of which may include; metals such as sodium, magnesium, lithium, potassium, aluminium, indium, silver, lead, chromium and their alloys, or oxides. In the case of metal oxide, those such as indium oxide (ITO), indium zinc oxide (IZO) and zinc oxide are suitable.

The insulating layer may be deposited adjacent to either electrode to avoid current leakage as mentioned in embodiments (3) to (8). As the insulating material, it is preferred to use an inorganic compound, examples of which may include aluminium oxide, lithium fluoride, lithium oxide, caesium oxide, magnesium oxide, magnesium fluoride, calcium oxide, calcium fluoride, aluminium nitride, titanium oxide, silicon oxide, silicon nitride, boron nitride, vanadium oxide.

The substrate (e.g., 1 shown in FIGS. 1-4) for the organic electroluminescence device of the present invention may include an opaque substrate made from any suitable material, such as metal or ceramics, or a transparent substrate made from any suitable transparent material such as glass, quartz, plastics, etc. It is possible to form the substrate with a colour filter film, a fluorescent colour conversion film, dielectric reflection film, etc., thus controlling such aspects of the emitted luminescent light.

The devices of the present application can be provided in the form of a stacked organic electroluminescent (EL) device. The present application also extends to electronic devices comprising the organic electroluminescent device of the present invention, including displays and light sources.

The present invention will be described below in detail with preparation examples and the device examples, but the present invention is not intended to be restricted to these examples.

### EXAMPLE 1

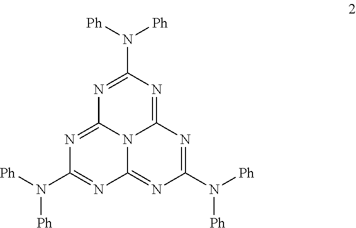

### Hexaphenylmelem 2

Following the procedure described by Schroeder and Kober (Schroeder, H., Kober, E. J. Org. Chem. 1962, 27, 4262.), a mixture of cyameluric chloride (2.30 g, 8.34 mmol) prepared by the known procedure (Schwarz, M., Buschmann, H. H., Kroke, E., Jiménez Alonso, O., Holenz, J., Corbera Arjona, J., Payella, D., Pelejero, C., Trullas, C-R. 2007, EP patent 1854797A1.) and Ph₂NH (9.87 g, 58.4 mmol) in xylenes (100 mL) was heated at 80° C. for 14 h. The resulting salmon coloured mixture was collected on a sintered glass funnel to give a solid residue. The residue was washed with H₂O (two times) and then EtOH (two times). The residue was taken up in CH₂Cl₂, filtered and concentrated to give a solid residue. The residue was purified by flash chromatography (MeOH/CH₂Cl₂, gradient; 0:100, 2:98, 3:97 then 4:96) to yield hexaphenylmelem 2 (3.89 g, 69%) as a colourless solid. A portion of this material was further purified by, firstly, recrystallisation (nitromethane) and, secondly, by sublimation (350° C., 0.025 mBar): m.p. 458-465° C. (DSC); R_{f}0.30 (5:95 EtOAc/CH₂Cl₂); ¹H NMR (CD₂Cl₂, 400 MHz) δ 7.22-7.29 (m, 18H), 7.34-7.40 (m, 12H); ¹³C NMR (CD₂Cl₂, 100 MHz) δ 127.99, 128.28, 129.96, 143.29, 156.44, 165.26; HRMS (EI) m/z 673.2589 C₄₂H₂₉N₁₀ [M−H]^{+•} requires 673.2571.

### EXAMPLE 2

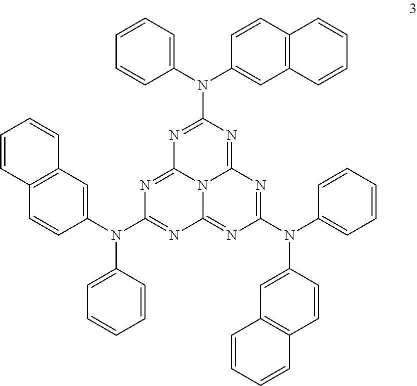

### N′,N″,N′″-Triphenyl-N′,N″,N′″-(tri-2-naphthyl)melem 3

A mixture of cyameluric chloride (2.46 g, 8.90 mmol) and N-phenyl-2-naphthylamine (13.6 g, 62.1 mmol) in xylenes (100 mL) was heated at 80° C. for 20 h. The resulting red coloured mixture was collected on a sintered glass funnel to give a solid residue. The residue was washed with H₂O (two times) and then EtOH (two times). The residue was taken up in CH₂Cl₂, filtered and concentrated to give a solid residue. The residue was purified by flash chromatography (EtOAc/CH₂Cl₂, gradient; 0:100, 1:99, 2:98 then 3:97) to yield N′,N″,N′″-triphenyl-N′,N″,N′″-(tri-2-naphthyl)melem 3 (1.51 g, 20%) as a pale yellow solid. A portion of this material was further purified by, firstly, recrystallisation (CH₂Cl₂/hexanes) and, secondly, by sublimation (350° C., 0.025 mBar): m.p. 417-425° C. (DSC); R_{f}0.37 (3:97 EtOAc/CH₂Cl₂); ¹H NMR (CDCl₃, 400 MHz) δ 7.11-7.32 (m, 15H), 7.34-7.40 (m, 9H), 7.54-7.58 (m, 3H), 7.63-7.68 (m, 3H), 7.70-7.77 (m, 6H); ¹³C NMR (CDCl₃, 100 MHz) δ 126.11, 126.26, 126.31, 126.90, 127.65, 127.88, 128.78, 129.09, 131.84, 133.47, 140.39, 142.85, 155.93, 164.50; HRMS (EI) m/z 823.3067 C₅₄H₃₅N₁₀ [M−H]^{+•} requires 823.3041.

### EXAMPLE 3

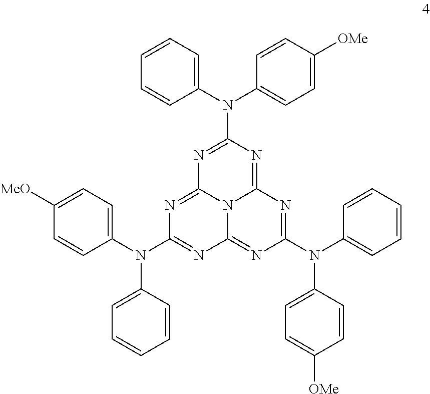

### N′,N″,N′″-Triphenyl-N′,N″,N′″-(tri-4-methoxyphenyl)melem 4

A mixture of cyameluric chloride (831 mg, 3.01 mmol) and N-4-methoxyphenyl-phenylamine (4.19 g, 21.1 mmol) in xylenes (50 mL) was heated at 80° C. for 22 h. The resulting dark coloured mixture was collected on a sintered glass funnel to give a solid residue. The residue was washed with H₂O (two times) and then EtOH (two times). The residue was taken up in CH₂Cl₂, filtered and concentrated to give a solid residue. The residue was purified by flash chromatography (EtOAc/CH₂Cl₂, gradient; 0:100, 2.5:97.5 then 4:96) to yield N′,N″,N′″-triphenyl-N′,N″,N′″-(tri-4-methoxyphenyl)melem 4 (1.68 g, 73%) as a pale orange solid. A portion of this material was further purified by, firstly, recrystallisation (CH₂Cl₂/toluene) and, secondly, by sublimation (350° C., 0.025 mBar): m.p. 487-496° C. (DSC); R_{f} 0.17 (5:95 EtOAc/CH₂Cl₂); ¹H NMR (CD₂Cl₂, 400 MHz) δ 3.77 (s, 9H), 6.88 (d, J=8.8 Hz, 6H), 7.16 (d, J=8.8 Hz, 6H), 7.20-7.29 (m, 9H), 7.33-7.40 (m, 6H); ¹³C NMR (CD₂Cl₂, 100 MHz) δ 55.91, 115.20, 127.82, 128.07, 129.26, 129.92, 136.05, 143.52, 159.22, 165.37; HRMS (EI) m/z 763.2891 C₄₅H₃₅N₁₀O₃ [M−H]^{+•} requires 763.2888.

### EXAMPLE 4

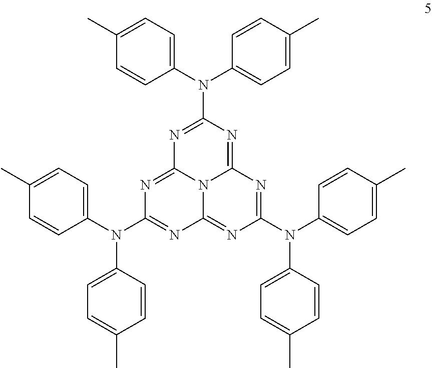

### Hexa-4-tolylmelem 5

A mixture of cyameluric chloride (1.93 g, 6.99 mmol) and di-4-tolylamine (9.60 g, 48.7 mmol) in xylenes (100 mL) was heated at 80° C. for 15 h. The resulting dark coloured mixture was collected on a sintered glass funnel to give a solid residue. The residue was washed with H₂O (two times) and then EtOH (two times). The residue was taken up in CH₂Cl₂, filtered and concentrated to give a solid residue. The residue was purified by flash chromatography (EtOAc/CH₂Cl₂/hexanes, gradient; 0:50:50, 2.5:48.75:48.75 then 2.5:72.5:25) to yield hexa-4-tolylmelem 5 (4.47 g, 84%) as a colourless solid. A portion of this material was further purified by, firstly, recrystallisation (CH₂Cl₂/toluene) and, secondly, by sublimation (350° C., 0.025 mBar): m.p. 459-468° C. (DSC); R_{f}0.59 (3:97 EtOAc/CH₂Cl₂); ¹H NMR (CDCl₃, 400 MHz) δ 2.28 (s, 18H), 7.01-7.10 (m, 24H); ¹³C CNMR (CDCl₃, 100 MHz) δ 21.04, 127.38, 129.70, 136.57, 140.25, 155.40, 163.90; HRMS (EI) m/z 757.3537 C₄₈H₄₁N₁₀ [M−H]^{+•} requires 757.3510.

### EXAMPLE 5

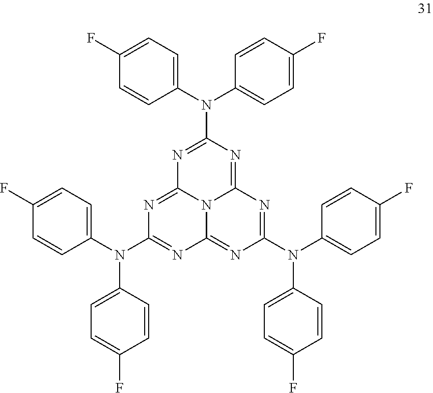

### Hexa-(4-fluorophenyl)melem 31

To a solution of bis(4-fluorophenyl)amine (2.81 g, 13.7 mmol) in THF (75 mL) at 0° C. was added NaH (60% dispersion in mineral oils, 575 mg, 14.3 mmol) and the resulting deep red mixture was stirred (10 min). Cyameluric chloride (841 mg, 3.05 mmol) was added and the mixture was stirred for 15 h at room temperature and then heated to reflux for 3 h. The reaction was allowed to cool to room temperature and saturated aqueous NH₄Cl (3 mL) was added and the reaction mixture was concentrated to ˜10 mL. The mixture was taken up in H₂O and collected on a sintered glass funnel to give a solid residue. The residue was washed with H₂O (two times) and then EtOH (three times). The residue was taken up in CH₂Cl₂, filtered and pre-adsorbed onto silica. This pre-adsorbed material was subjected to flash chromatography (EtOAc/CH₂Cl₂, gradient; 1:99 to 4:96) to yield hexa-(4-fluorophenyl)melem 31 (1.5 g, 63%) as a colourless solid. A portion of this material was further purified by, firstly, recrystallisation (CH₂Cl₂/toluene) and, secondly, by sublimation (310° C., 10⁻⁵ mBar): m.p. 493-496° C. (DSC); ¹H NMR (CD₂Cl₂, 400 MHz) δ 7.04-7.11 (m, 12H), 7.18-7.24 (m, 12H); HRMS (EI) m/z 781.2033 C₄₂H₂₃N₁₀F₆ [M−H]^{+•} requires 781.2006.

### EXAMPLE 6

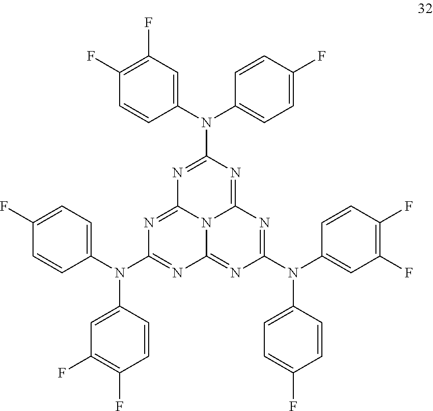

### N′,N″,N′″-(Tri-3,4-difluorophenyl)-N′,N″,N′″-(tri-4-fluorophenyl)melem 32

To a solution of 3,4-difluoro-N-(4-fluorophenyl)aniline (1.85 g, 8.3 mmol) in THF (30 mL) at 0° C. was added NaH (60% dispersion in mineral oils, 350 mg, 8.66 mmol) and the resulting mixture was stirred at room temperature (20 min) and then at 50° C. (20 min). Cyameluric chloride (510 mg, 1.84 mmol) was added and the mixture was heated to reflux for 8 h. The reaction was allowed to cool to room temperature and saturated aqueous NH₄Cl (3 mL) was added and the reaction mixture was concentrated to ˜10 mL. The mixture was diluted with CH₂Cl₂ and H₂O and the organic phase was separated. The aqueous phase was re-extracted (CH₂Cl₂) and the combined organics were washed (saturated aqueous NaCl), dried (MgSO₄), filtered and concentrated to give a solid residue. The residue was purified by flash chromatography (EtOAc/hexanes/CH₂Cl₂ 0:25:75 then 1:25:74 then 2.5:25:72.5) to yield N′,N″,N′″-tri-3,4-difluorophenyl)-N′,N″,N′″-(tri-4-fluorophenyl)melem 32 (625 mg, 40%) as a colourless solid. A portion of this material was further purified by, firstly, recrystallisation (CH₂Cl₂/toluene) and, secondly, by sublimation (310° C., 10⁻⁵ mBar): m.p. 424-427° C. (DSC); ¹H NMR (CD₂Cl₂, 400 MHz) δ 6.97-7.05 (m, 3H), 7.06-7.14 (m, 9H), 7.15-7.36 (m, 9H); ¹³C NMR (CD₂Cl₂, 100 MHz) δ 117.00, 117.23, 117.84 (d, J 19 Hz), 118.40 (d, J 19 Hz), 124.77 (d, J4 Hz), 129.97, 130.06, 138.58 (d, J 3 Hz), 139.19 (q, J 3 Hz), 149.22 (dd, J 14, 73 Hz), 151.64 (dd, J 14, 73 Hz), 156.58, 162.16 (J 247 Hz), 165.46; HRMS (ESI) m/z 837.1891 C₄₂H₂₂N₁₀F₉ [M+H]^{+•} requires 837.1885.

### EXAMPLE 7

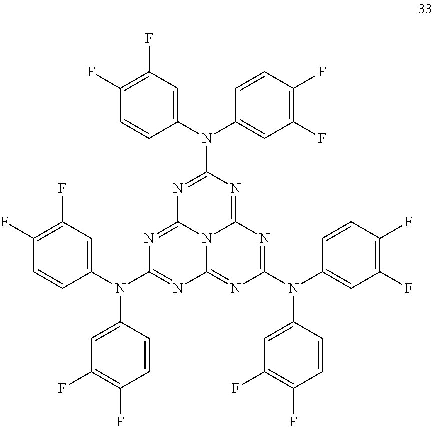

### Hexa-(3,4-difluorophenyl)melem 33

To a solution of bis(3,4-difluorophenyl)amine (2.95 g, 12.2 mmol) in THF (70 mL) was added NaH (60% dispersion in mineral oils, 490 mg, 12.8 mmol) and the resulting mixture was stirred at room temperature (30 min). Cyameluric chloride (750 mg, 2.72 mmol) was added and the mixture was heated to reflux for 20 h. The reaction was allowed to cool to room temperature and saturated aqueous NH₄Cl (3 mL) was added and the reaction mixture was concentrated to ˜10 mL. The mixture was diluted with CH₂Cl₂ and H₂O and the organic phase was separated. The aqueous phase was re-extracted (CH₂Cl₂) and the combined organics were washed (saturated aqueous NaCl), dried (MgSO₄), filtered and concentrated to give a solid residue. The residue was purified by flash chromatography (EtOAc/CH₂Cl₂ 0:100 then 1:99 then 2:98) to yield hexa-(3,4-difluorophenyl)melem 33 (1.1 g, 45%) as a colourless solid. A portion of this material was further purified by recrystallisation (CH₂Cl₂/toluene): m.p. 389-393° C. (DSC); ¹H NMR (CDCl₃, 400 MHz) δ 6.87-6.93 (m, 6H), 7.02 (ddd, J 2.6, 6.9, 10.7 Hz, 6H), 7.11 (q, J 8.8 Hz, 6H); ¹³C NMR (CDCl₃, 100 MHz) δ 117.34, (d, J 19 Hz), 117.66 (d, J 19 Hz), 124.03 (d, J 4 Hz), 138.03 (q, J 4 Hz), 148.48 (dd, J 13, 82 Hz), 150.98 (dd, J 14, 82 Hz), 156.07, 164.57; LRMS (EI) m/z 889.4 C₄₂H₁₇N₁₀F₁₂ [M−H]^{+•} requires 889.1.

### EXAMPLE 8

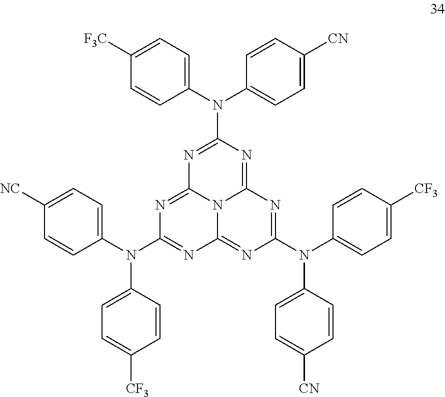

### N′,N″,N′″-(Tri-4-cyanophenyl)-N′,N″,N′″-(tri-4-trifluoromethylphenyl)melem 34

To a solution of 4-(4-(trifluoromethyl)phenylamino)benzonitrile (1.6 g, 6.11 mmol) in THF (120 mL) at 0° C. was added NaH (60% dispersion in mineral oils, 260 mg, 6.40 mmol) and the resulting deep red mixture was stirred (10 min). Cyameluric chloride (420 mg, 1.52 mmol) was added and the mixture was heated to reflux for 20 h. The reaction was allowed to cool to room temperature and saturated aqueous NH₄Cl (3 mL) was added and the reaction mixture was concentrated to ˜10 mL. The mixture was diluted with CH₂Cl₂ and H₂O and the organic phase was separated. The aqueous phase was re-extracted (CH₂Cl₂) and the combined organics were washed (saturated aqueous NaCl), dried (MgSO₄), filtered and concentrated to give a solid residue. The residue was purified by flash chromatography (EtOAc/hexanes, 30:70 then 0:100 then EtOAc/CH₂Cl₂ 5:95) to yield N′,N″,N′″-(tri-4-cyanophenyl)-N′,N″,N′″-(tri-4-trifluoromethylphenyl)melem 34 (610 mg, 42%) as a colourless solid. A portion of this material was further purified by, firstly, recrystallisation (CH₂Cl₂/toluene) and, secondly, by sublimation (350° C., 10⁻⁵ mBar): m.p. 484-489° C. (DSC); ¹H NMR (CDCl₃, 400 MHz) δ 7.32 (t, J 9 Hz 12H), 7.67 (d, J 9.0 Hz, 12H); ¹³C NMR (CDCl₃, 100 MHz) δ 112.01, 118.44, 124.24 (q, J 270 Hz), 127.38 (q, J 14 Hz), 128.94, 129.11, 130.26 (q, J 33 Hz), 133.98, 145.18, 146.13, 156.88, 165.27; HRMS (ESI) m/z 976.2044 C₄₈H₂₄N₁₅NaF₉ [M+Na]^{+•} requires 976.2032.

### EXAMPLE 9

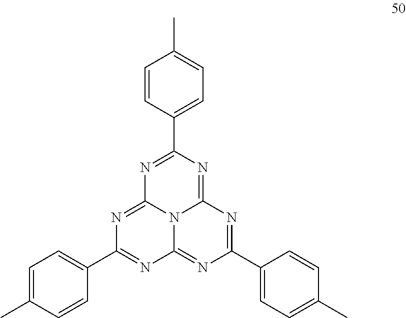

### Tris-(4-tolyl)-tri-s-triazine 50

A mixture of AlCl₃ (3.32 g, 25 mmol) in toluene (8.5 mL) was heated to 65° C. Cyameluric chloride (1.5 g, 5.4 mmol) was added in six portions over 30 min. This mixture was stirred at 65° C. for 5 h and then quenched with the addition of H₂O (30 mL) and stirred vigorously to afford a flocculent yellow precipitate. The reaction mixture was filtered and the yellow precipitate was washed (three times) with H₂O. The yellow precipitate was taken up in toluene (˜700 mL) and pre-adsorbed onto silica. This pre-adsorbed material was subjected to flash chromatography (EtOAc/toluene, gradient; 0:99 to 2.5:97.5) to yield tris-(4-tolyl)-tri-s-triazine 50 as a yellow solid (540 mg, 22%). This material was further purified by, firstly, recrystallisation (xylenes) and, secondly, by sublimation (280° C., 10⁻⁵ mBar): m.p. 449-458° C. (DSC); ¹H NMR (C₆D₆, 400 MHz) δ 2.03 (s, 9H), 7.04 (d, J 8.0 Hz, 6H), 8.80 (d, J 8.2 Hz, 6H); HRMS (EI) m/z 443.1855 C₂₇H₂₁N₇ [M]^{+•} requires 443.1853.

### EXAMPLE 10

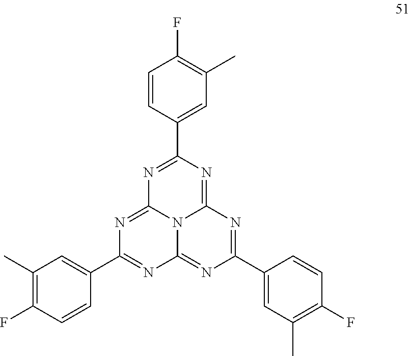

### Tris-(4-fluoro-3-methylphenyl)-tri-s-triazine 51

To a mixture of AlCl₃ (2.41 g, 18 mmol) in 2-fluorotoluene (8.5 mL) at 0° C. was added cyameluric chloride (1.0 g, 3.6 mmol) and the mixture was stirred at 0° C. for 30 min and then at 80° C. for 4 h. The reaction was quenched with the addition of ice and the mixture was stirred vigorously at room temperature for 1 h and then at 100° C. for 30 min to afford a flocculent yellow precipitate. The reaction mixture was allowed to cool to room temperature, filtered and the yellow precipitate was washed (three times) with H₂O and then taken up in toluene (˜500 mL) and pre-adsorbed onto silica. This pre-adsorbed material was subjected to flash chromatography (EtOAc/toluene, gradient; 0:100 to 2:98) to yield tris-(4-fluoro-3-methylphenyl)-tri-s-triazine 51 as a yellow solid (1.03 g, 57%). This material was further purified by, firstly, recrystallisation (xylenes) and, secondly, by sublimation (265° C., 10⁻⁵ mBar): m.p. 370-380° C. (DSC); ¹H NMR (C₆D₆, 400 MHz) δ 2.06 (s, 9H), 6.81 (t, J 8.8 Hz, 3H), 8.54-8.61 (m, 3H), 8.65 (d, J 7.6 Hz, 3H); HRMS (EI) m/z 497.1580 C₂₇H₁₈N₇F₃ [M]^{+•} requires 497.1570.

### EXAMPLE 11

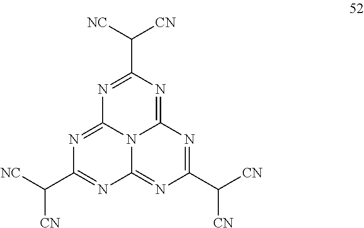

### Tris-(dicyanomethyl)-tri-s-triazine 52

To malononitrile (980 mg, 14.8 mmol) in THF (30 mL) at 0° C. was added (in portions over 5 min) NaH (60% dispersion in mineral oils, 610 mg, 15.3 mmol) and the mixture was stirred (30 min). Cyameluric chloride (580, 2.12 mmol) was added and the mixture was heated to reflux for 8 h. The mixture was allowed to cool to room temperature and saturated aqueous NH₄Cl (3 mL) was added and the reaction mixture was concentrated to ˜10 mL. H₂O was added and a precipitate formed. The mixture was filtered and the solid washed with H₂O (3 times). The solid was extracted exhaustively with EtOAc and this extract was pre-adsorbed onto C18 reverse phase silica. This pre-adsorbed material was subjected to C18 reverse phase silica chromatography (MeCN/H₂O, gradient; 0:100 to 15:85) to yield tris-(dicyanomethyl)-tri-s-triazine 52 as a yellow solid (400 mg, 52%): ¹³C NMR (CD₃)₂SO, 100 MHz) δ 49.49, 117.07, 146.53, 165.35; LRMS (EI) m/z 889.4 C₁₅H₃N₁₃ [M+H]^{+•} requires 366.3.

### EXAMPLE 12

The hole injection property of the example compounds was confirmed by making device comprised of two layers using an example compound and NPD (N,N′-di(1-naphthyl)-N,N′-diphenyl-(1,1′-biphenyl)-4,4′-diamine). The device was manufactured as described below;

Device 1; Compound 2 having 4 nm thickness was deposited on an aluminium film (of 1000 Å) on a glass substrate under 10⁻⁶ torr. NPD having a 100 nm thickness was deposited onto the compound 2 layer under the same conditions. Finally, aluminium film having 1000 Å thickness was deposited onto the NPD layer.

Device 2: Device 2 was fabricated using compound 5 instead of compound 2 under the same conditions as above.

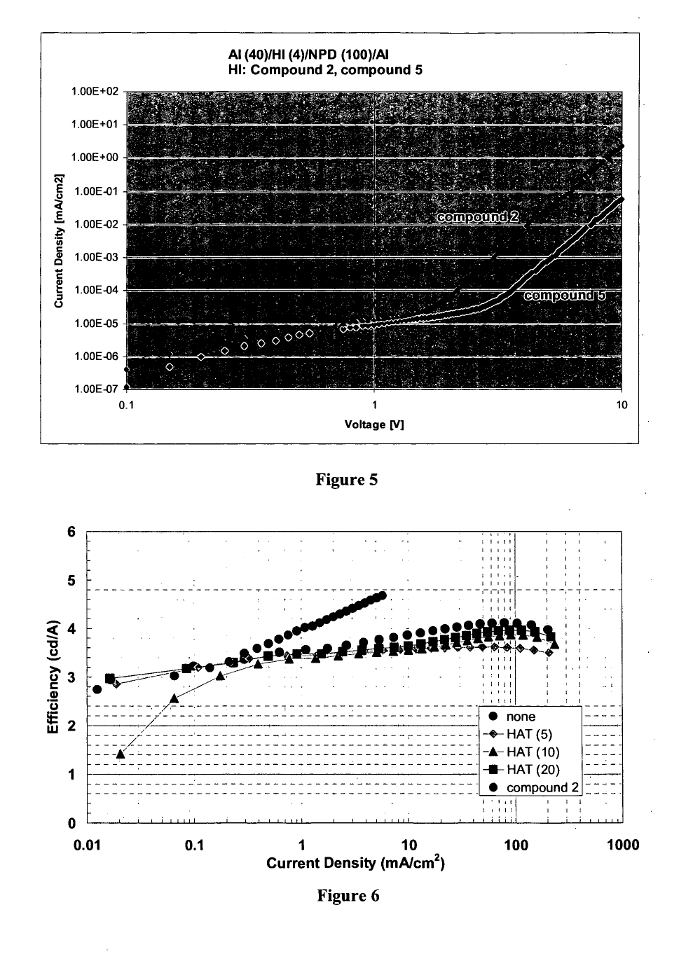

The device performance data is shown in FIG. 5. The hole current was observed even at low driving voltage.

### EXAMPLE 13

Devices 3, 4, 5, 6 and 7 using Aluminium quinolate (Alq) as electron transporting material were fabricated to compare the power efficiency.

The layer structure for devices 3-7 is; Glass /ITO(130 nm)/Hole Injection(x)/NPD(50 nm)/Alq(70 nm)/Al(100 nm). All layers were vacuum deposited.

Device 3: Comprises no Hole Injection layer.

Device 4: Comprises Hexacyanohexaazatriphenylene (HAT) (5 nm thickness as a Hole Injection layer).

Device 5: Comprises Hexacyanohexaazatriphenylene (HAT) (10 nm thickness as a Hole Injection layer).

Device 6: Comprises Hexacyanohexaazatriphenylene (HAT) (20 nm thickness as a Hole Injection layer).

Device 7: Comprises Compound 2 (3 nm thickness as a Hole Injection layer).

The device performance data is shown in FIG. 6.

1. An organic electroluminescent device comprising:
a pair of electrodes comprising an anode and a cathode, and
one or more layers of organic compound arranged between the pair of electrodes, wherein the organic compound layer, or one or more of the organic compound layers, comprises a compound represented by the following formula (1): 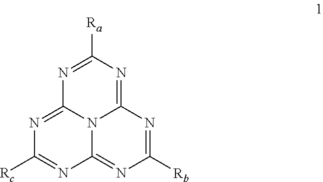
wherein:
Rₐ is selected from NR₁R₂, CR₇R₈R₉ and Ar₁₆;
R_{b} is selected from NR₃R₄, CR₁₀R₁₁R₁₂ and Ar₁₇;
R_{c} is selected from NR₅R₆, CR₁₃R₁₄R₁₅ and Ar₁₈;
R₁ to R₆ may be the same or different, and are each independently selected from the group consisting of substituted aryl groups and substituted or unsubstituted heterocyclic groups;
R₇, R₁₀ and R₁₃ are each hydrogen;
R₈, R₉, R₁₁, R₁₂, R₁₄ and R₁₅ may be the same or different, and are each independently substituted aryl groups containing one or more substituents selected from the group consisting of halogen, CN and NO₂, or combinations or multiples thereof, or substituted heterocyclic groups containing one or more substituents selected from the group consisting of halogen, CN and NO₂, or combinations or multiples thereof; and
Ar₁₆ to Ar₁₈ are each selected from substituted aryl groups in which the substituent may contain a monomer or a polymer chain.

2. A device according to claim 1, wherein when R₁ to R₆ is a substituted aryl group or Ar₁₆ to Ar₁₈ are substituted aryl groups, the one or more substituents on the aryl group are selected from the group consisting of alkyl, halogen, cyano and nitro, or combinations or multiples thereof.

3. A device according to claim 1, wherein the compound is of formula (1a): 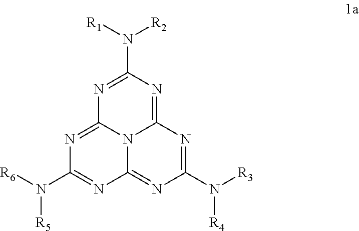
wherein:
R₁ to R₆ may be the same or different, and are each independently selected from the group consisting of substituted aryl groups and substituted or unsubstituted heterocyclic groups.

4. A device according to claim 3, wherein the aryl group is monocyclic.

5. A device according to claim 3, wherein the aryl group is polycyclic.

6. A device according to claim 3, wherein when R₁ to R₆ is a substituted aryl group, the one or more substituents on the aryl group are selected from the group consisting of alkyl, halogen, cyano and nitro, or combinations or multiples thereof.

7. A device according to claim 3, wherein R₁ to R₆ are each independently selected from substituted or unsubstituted heterocyclic groups.

8. A device according to claim 7, wherein the heterocyclic groups are polyheterocyclic.

9. A device according to claim 7, wherein the heterocyclic group is selected from carbazole, pyridine, quinoline, pyrazole, pyrazoline, pyrazine, triazole, isoxazole, oxazine, thiazine, oxazole, imidazole, oxadiazole, furan, thiophene, pyrrole, phenothiazine, phenoxazine, pyrazolidine.

10. A device according to claim 3, wherein each of R₁ to R₆ is the same.

11. A device according to claim 3, wherein each of R₁, R₃ and R₅ are the same, and each of R₂, R₄ and R₆ is the same, but R₁ is different to R₂.

12. A device according to claim 1, wherein the compound is of formula (1b): 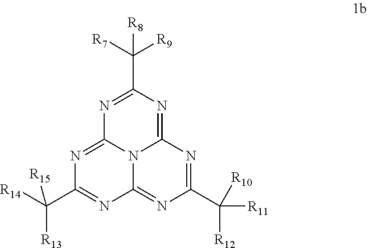
wherein:
R₇, R₁₀ and R₁₃ are each hydrogen;
R₈, R₉, R₁₁, R₁₂, R₁₄ and R₁₅ may be the same or different, and are each independently substituted aryl groups containing one or more substituents selected from the group consisting of halogen, CN and NO₂, or combinations or multiples thereof, or substituted heterocyclic groups containing one or more substituents selected from the group consisting of halogen, CN and NO₂, or combinations or multiples thereof.

13. A device according to claim 1, wherein the compound is of formula (1c): 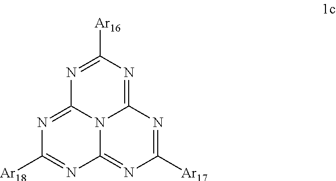
wherein:
Ar₁₆ to Ar₁₈ are each selected from substituted aryl groups in which the substituents may contain a monomer or polymer chain.

14. A device according to claim 1, wherein the device is a stacked organic EL device.

15. A device according to claim 1, wherein the device comprises a display.

16. A device according to claim 1, wherein the device comprises a light source.

17. A compound of formula (1): 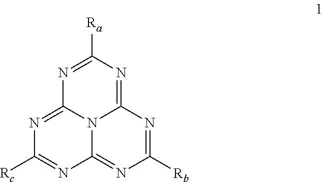
wherein:
Rₐ is selected from NR₁R₂, CR₇R₈R₉ and Ar₁₆;
R_{b} is selected from NR₃R₄, CR₁₀R₁₁R₁₂ and Ar₁₇;
R_{c} is selected from NR₅R₆, CR₁₃R₁₄R₁₅ and Ar₁₈,
R₁ to R₆ may be the same or different, and are each independently selected from the group consisting of substituted aryl groups and substituted heterocyclic groups;
R₇, R₁₀ and R₁₃ are each hydrogen;
R₈, R₉, R₁₁, R₁₂, R₁₄ and R₁₅ may be the same or different, and are each independently substituted aryl groups containing one or more substituents selected from the group consisting of halogen, CN and NO₂, or combinations or multiples thereof, or substituted heterocyclic groups containing one or more substituents selected from the group consisting of halogen, CN and NO₂, or combinations or multiples thereof; and
Ar₁₆ to Ar₁₈ are each selected from substituted aryl groups containing one or more substituents selected from the group consisting of halogen, CN and NO₂, or combinations or multiples thereof in which the substituents may contain a monomer or polymer chain.

18. A compound of formula (1a): 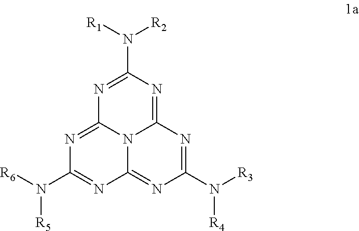
wherein:
R₁ to R₆ may be the same or different, and are each independently selected from the group consisting of substituted aryl groups containing one or more substituents selected from the group consisting of halogen, CN and NO₂, or combinations or multiples thereof and substituted heterocyclic groups.

19. A compound of formula (1b): 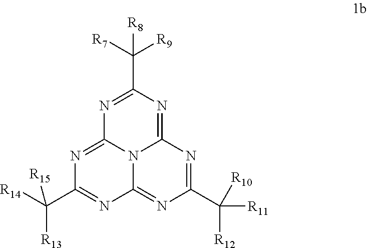
wherein:
R₇ , R₁₀ and R₁₃ are each hydrogen;
R₈, R₉, R₁₁, R₁₂, R₁₄ and R₁₅ may be the same or different, are each independently substituted aryl groups containing one or more substituents selected from the group consisting of halogen, CN and NO₂, or combinations or multiples thereof, or substituted heterocyclic groups containing one or more substituents selected from the group consisting of halogen, CN and NO₂, or combinations or multiples thereof.

20. A compound of formula (1c): 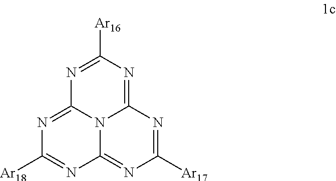
wherein:
Ar₁₆ to Ar₁₈ are each selected from substituted aryl groups containing one or more substituents selected from the group consisting of halogen, CN and NO₂, or combinations or multiples thereof in which the substituents may contain a monomer or polymer chain.

21. Use of a compound of formula (1) 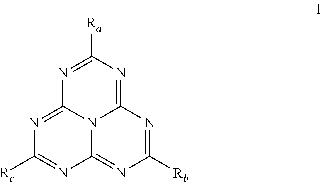
wherein:
Rₐ is selected from NR₁R₂, CR₇R₈R₉ and Ar₁₆;
R_{b} is selected from NR₃R₄, CR₁₀R₁₁R₁₂ and Ar₁₇;
R_{c} is selected from NR₅R₆, CR₁₃R₁₄R₁₅ and Ar₁₈;
R₁ to R₆ may be the same or different, and are each independently selected from the group consisting of substituted aryl groups and substituted or unsubstituted heterocyclic groups;
R₇, R₁₀ and R₁₃ are each hydrogen;
R₈, R₉, R₁₁, R₁₂, R₁₄ and R₁₅ may be the same or different, are each independently substituted aryl groups containing one or more substituents selected from the group consisting of halogen, CN and NO₂, or combinations or multiples thereof, or substituted heterocyclic groups containing one or more substituents selected from the group consisting of halogen, CN and NO₂, or combinations or multiple thereof; and
Ar₁₆ to Ar₁₈ are each selected from substituted aryl groups in which the substituents may contain a monomer or polymer chain in an organic electroluminescent device.

22. Use of a compound of formula (1) 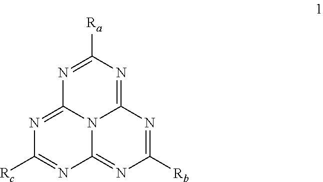
wherein:
Rₐ is selected from NR₁R₂, CR₇R₈R₉ and Ar₁₆;
R_{b} is selected from NR₃R₄, CR₁₀R₁₁R₁₂ and Ar₁₇;
R_{c} is selected from NR₅R₆, CR₁₃R₁₄R₁₅ and Ar₁₈;
R₁ to R₆ may be the same or different, and are each independently selected from the group consisting of substituted aryl groups and substituted or unsubstituted heterocyclic groups;
R₇, R₁₀ and R₁₃ are each hydrogen;
R₈, R₉, R₁₁, R₁₂, R₁₄ and R₁₅ may be the same or different, are each independently substituted aryl groups containing one or more substituents selected from the group consisting of halogen, CN and NO₂, or combinations or multiples thereof, or substituted heterocyclic groups containing one or more substituents selected from the group consisting of halogen, CN and NO₂, or combinations or multiple thereof; and
Ar₁₆ to Ar₁₈ are each selected from substituted aryl groups in which the substituents may contain a monomer or polymer chain, as a hole injection material, a hole generation material, a hole injection layer, a hole generation layer, or as a dopant in a hole transporting layer.
